(19) United States
(12) Patent Application Publication
(10) Pub. No.: US 2020/0229760 A1
(43) Pub. Date: Jul. 23, 2020

(54) Sensor and Feedback Platform for Use in Orthotic and Prosthetic Devices

(71) Applicant: Wellinks, Inc., New Haven, CT (US)

(72) Inventors: Levi DeLuke, New Haven, CT (US); Ellen Su, San Francisco, CA (US); Sebastian Monzon, New Haven, CT (US)

(73) Assignee: Wellinks, Inc., New Haven, CT (US)

(21) Appl. No.: 16/586,233

(22) Filed: Sep. 27, 2019

Related U.S. Application Data
(63) Continuation of application No. 15/142,151, filed on Apr. 29, 2016, now abandoned.
(60) Provisional application No. 62/272,141, filed on Dec. 29, 2015.
(60) Provisional application No. 62/154,393, filed on Apr. 29, 2015.

(51) Int. Cl. A61B 5/00; A61F 5/02; A61F 5/01; A61F 5/37; A61F 2/60; G01B 7/24

(52) U.S. Cl. CPC A61B 5/4851; A61F 5/02; A61F 5/0123; A61F 5/0127; A61F 2220/0008; A61F 2/60; G01B 7/24; A61F 2250/0096; A61F 5/3738

(57) ABSTRACT

Systems and methods for monitoring/measuring parameters related to the use of devices/systems in various diagnostic/therapeutic applications are provided. The systems/methods communicate monitored/measured parameters to data processing and/or data display units for review and/or responsive action. Modular units may be provided for use with orthotic devices, e.g., arm slings and orthotic boots for foot and lower leg immobilization, and in conjunction with prosthetic devices, e.g., prosthetic arms and/or legs. The sensing/feedback mechanisms may be strap-based, or mounted/associated with webbing, ratchet systems and/or other tensioning mechanisms. The sensing/feedback mechanism may include an inductive sensor that interacts with conductive material embedded in a strap to produce a signal indicating the location of the inductive sensor with respect to the strap. The position sensor may include multiple coils and multiple conductive materials may be imbedded in the strap. The conductive material may define a variable width along the X-axis (as defined by the strap). The inductive sensor may also be used to measure the distance from the coil and a conductive material that interacts with a section of the orthotic or prosthetic device along the z-axis.

### CROSS-REFERENCE TO RELATED APPLICATIONS

The present application is a continuation patent application claiming priority benefit to a U.S. non-provisional application entitled “Sensor and Feedback Platform for Use in Orthotic and Prosthetic Devices,” which was filed on Apr. 29, 2016 and assigned Ser. No. 15/142,151, and which claimed priority benefit to two (2) provisional patent applications: (i) a first provisional patent application entitled “Sensor and Feedback Platform for Use in Orthotic and Prosthetic Devices”, filed on Apr. 29, 2015, and designated by Ser. No. 62/154,393, and (ii) a second provisional patent application entitled “Position Sensing Using an Inductive Sensor for Brace-Based Equipment”, filed on Dec. 29, 2015, and designated by Ser. No. 62/272,141. The entire contents of the foregoing non-provisional patent application and both the first and the second provisional applications are incorporated herein by reference.

### BACKGROUND

### Technical Field

The present disclosure is directed to the use of sensing systems and methods for monitoring and/or measuring parameters related to devices/systems for use in various diagnostic, therapeutic and/or prosthetic applications, and to communicating the monitored and/or measured parameters to data processing and/or data display units for review and/or responsive action. Exemplary implementations of the disclosed systems and methods relate to modular attachment apparatus/mechanisms for use with orthotic braces and prosthetic devices.

### Background Art

The use of braces, e.g., scoliosis braces, orthotic braces and prosthetic devices, to correct and/or limit further damage or degradation to orthotic conditions has been long-standing. For example, scoliosis braces, leg braces and arm slings are frequently used to immobilize limbs that have incurred bone or soft-tissue injury. In another example, prosthetic limbs can vastly improve, or even return the quality of life to those who have sustained injuries that resulted in loss of limbs. Current treatment methods, whether orthotic braces or prosthetic devices, frequently use strap systems that require an optimum tension to facilitate proper fit and/or healing. Of note, in some cases the orthotic braces and, in particular, prosthetic devices are custom fabricated for each individual patient based on unique anatomical considerations. The information supplied to the user and/or the users' colleague(s), e.g., parent(s), is limited in terms of the use of the brace or device. Indeed, users and others involved in assisting users of such braces/devices are frequently uncertain as to whether the brace/device is being worn properly, e.g., tightened to an appropriate degree, or for an appropriate duration. As a result, care providers have no way to make informed decisions on patient wear characteristics in order to improve treatment.

In another example, adolescent idiopathic scoliosis is a medical condition characterized by a moderate to severe curvature of the spine. Current treatment methods may consist of a hard plastic brace that straightens the spine when the straps of the brace are tightened. Of note, each scoliosis brace is generally custom fabricated for each individual patient based on unique anatomical considerations. The information supplied to the user and/or the user's colleague(s), e.g., parent(s), is limited in terms of the use of the brace. Indeed, users and others involved in assisting users are frequently uncertain as to whether the brace is being worn properly, e.g., tightened to an appropriate degree, or for an appropriate duration

Use of prosthetic devices involves further challenges. Patients often find it hard to put on prosthetic devices correctly, in part because the devices often rely on the patients to tighten or otherwise don and doff the device. This difficulty can result in an uncomfortable fit and/or ineffective treatment. Patient reported data is necessary to adjust fit of prosthetics, or perhaps even change the type of device prescribed. Inaccurate information can result in improper fittings or prescriptions that will impact the patient's level of activity or adherence to therapy regimens. Health insurance reimbursement is often based on a patient's continued progress and could be in jeopardy without an appropriate or required level of adherence. Even when patients are able to correctly don and doff prescribed orthotic and/or prosthetic devices, they often have no way to keep track of and set goals for activity, steps, range of motion, to facilitate progress and/or recovery from an illness or injury.

There currently exists a gap between low-cost orthotics and prosthetics, which typically are purely mechanical devices with no electronics or sensing capabilities, and high end, expensive orthotics and prosthetics, which can provide a wealth of valuable information and feedback to users and care providers. Companies that would like to integrate sensing/feedback capabilities into their existing orthotic and prosthetic devices must often start from scratch, and are unable to utilize existing work in the field.

Furthermore, patients are currently told to pull the straps on their braces to a position that is prescribed by their orthotists. The point at which the strap is to be pulled is often marked with a marker and does not change in-between visits to the clinic. Currently there is not an effective method to measure this position automatically while the patients are away from the health care provider to ensure patients are pulling the strap to a correct point. It is hard for patients to pull to the point by themselves if the strap is behind their back, because they cannot see the mark drawn by the orthotist. Rather, the mark to which patients are to pull cannot be updated in-between visits resulting in a static target level for brace tightness.

Efforts have been made to develop compliance monitors for scoliosis braces, but commercially available efforts have failed to yield products/systems that meet the needs of users and/or medical professionals. For example, compliance monitors that have been developed-to-date suffer from shortcomings that include (i) an inability to incorporate or integrate the compliance monitor into existing brace designs, (ii) an inability to measure both compliance and quality of brace wear, and (iii) an inability to provide meaningful and/or actionable feedback to patients, colleagues of patients (e.g., parents) and/or physicians and other health care providers.

To the extent compliance monitors have been pursued, the focus-to-date (other than the work of the present inventors) has been directed to the incorporation of a temperature sensor to record how long a patient has worn the scoliosis brace. Thus, when the temperature sensor notes an elevated temperature, it is concluded that the scoliosis brace is being worn by the patient. Conversely, when an elevated temperature is absent, then it is concluded that the scoliosis brace is not being worn by the patient. As is readily apparent, the inclusion of a temperature sensor provides very limited information concerning a patient's use of a scoliosis brace. For example, no information is provided with respect to the quality of the brace's use, i.e., whether the brace is being properly worn. Moreover, the nature and quality of the information that is collected, analyzed and stored based on a temperature sensor provide little value to patients, colleagues of patients and/or physicians and other health care providers.

With reference to the patent literature, U.S. Pat. No. 6,926,667 to Khouri discloses a patient monitoring device that includes a microprocessor controller having a clock circuit and memory coupled to one or more sensors physically carried by a medical appliance, i.e., vacuum domes for enclosing the breasts of a female patient. According to the Khouri '667 patent, a pressure sensor may be provided in conjunction with one of the vacuum domes to confirm appropriate levels of negative pressure. A temperature sensor may be provided to confirm that a patient is wearing/using the medical device. A third sensor may be provided to confirm the information received from the first or second sensor. The sensors provide an electrical signal that may be timed to confirm a patient's compliance with a recommended protocol. By combining and correlating the sensor data with the clock or timer provided as part of the controller, a time chart of data may be created indicating when and for how long the patient actually wears the device.

U.S. Patent Publication No. 2009/0281469 to Conlon et al. discloses a compliance strapping that includes a predetermined adjustability, tamper deterring and indicating strapping, that is adapted, in use, to form an encircling loop. The compliance strapping is passed around an object and, for further security, the strap can be threaded through lining material or through a wearable article or medical device. The free end of the elongate member is passed through the loop, which may be a D-loop sewn into the strapping, thus forming an encircling loop of strapping. The second end is brought around to close proximity with a region of the strapping which has been passed through the loop. The tamper indicating means, referred to as a self-locking rivet, is fastened to this region of the strapping. Thus, the encircling loop cannot be broken because the region of the strapping with the self-locking rivet fastened thereto cannot pass back through the D-loop.

U.S. Pat. No. 6,540,707 to Stark et al. discloses an exercise orthosis that includes a frame, a fluid bladder held by the frame, a pressure sensor attached to the fluid bladder and a microprocessor for receiving pressure measurements from the pressure sensor. The microprocessor monitors variations in pressure and determines differences between the measured pressures and predetermined target values. The Stark '707 patent further discloses a corrective back orthosis that includes a frame, force applicators connected to the frame to apply force to the patient's spine, a sensor that measures forces associated with the force applicators, and a control unit that monitors forces measured by the sensor. The corrective back orthosis can include fluid bladders as force applicators and the control unit can include a microprocessor.

U.S. Pat. Nos. 6,890,285, 7,166,063 and 7,632,216 to Rahman et al. disclose brace compliance monitors. The Rahman patents generally disclose a brace compliance monitor that includes a compliance sensor, a signal processor, and a display. Compliance data from the Rahman systems is displayed on the display to provide the patient or subject with immediate compliance information on whether they have been wearing the brace for the specified period and in the specified manner. The brace compliance monitor may also include a secondary sensor, such as a tilt sensor, a pressure sensor, a force sensor, an acceleration sensor, or a velocity sensor. The secondary sensors may provide additional compliance data to the patient and health care provider.

Despite efforts to date, a need remains for systems and methods that effectively monitor and/or measure parameters related to the use of devices/systems in various diagnostic and/or therapeutic applications. In addition, a need remains for systems and methods that effectively communicate monitored and/or measured parameters that are collected from such devices/systems to data processing and/or data display units to facilitate review and/or responsive action. More specifically, a need remains for systems and methods that can effectively determine whether a device/system, e.g., an orthotic brace or a prosthetic device, is being properly used, both as to tightness and duration of use, and communicate this information so as to permit responsive action, whether in real-time or at a point in the future. Still further, a need remains for modular attachment mechanisms/modalities that allow broad-based application of advantageous monitoring and/or measuring functionalities across a range of diagnostic, therapeutic and/or prosthetic applications. These and other needs are satisfied by the systems and methods disclosed herein.

### SUMMARY

As noted above, the present disclosure is directed to applications of systems and methods for monitoring and/or measuring parameters related to the use of devices/systems in various diagnostic and/or therapeutic applications, and to communicating the monitored and/or measured parameters to data processing and/or data display units for review and/or responsive action. In exemplary embodiments, one or more modular units are provided for use in conjunction with orthotic devices, such as arm slings and orthotic boots for foot and lower leg immobilization. Additionally, exemplary embodiments of modular unit(s) for use in conjunction with prosthetic devices, e.g., prosthetic arms and/or legs, are provided.

The disclosed modular units generally include one or more sensing and/or feedback mechanisms integrated into or otherwise associated therewith. In exemplary implementations, the sensing/feedback mechanisms are strap-based, i.e., mounted or otherwise associated with strap(s) that interact with an orthotic and/or prosthetic device. However, alternative means of implementation relative to orthotic/prosthetic devices are contemplated, e.g., the disclosed modular unit(s) may be mounted or otherwise associated with webbing, ratchet systems and/or other tensioning mechanisms associate with an orthotic/prosthetic device.

In exemplary embodiments, the sensing/feedback unit may be embedded in or permanently fixed to the orthotic and/or prosthetic device. The sensing/feedback unit can sense information on the tightening mechanism or device state without directly being in line with or interacting mechanically with the strap, ratchet, or other tightening mechanism. using of sensing methods ,e.g., the inductive or magnetic methods described herein.

The sensing and/or feedback mechanisms associated with the disclosed modular units collect advantageous information as to use of the orthotic/prosthetic device, e.g., the quality and/or compliance of orthotic/prosthetic brace utilization by a prescribed user, step count, activity, range of motion, orientation, and/or additional metrics/measurements of interest. The underlying data collected by the disclosed sensing and/or feedback mechanisms may be the same and/or similar from application-to-application, but the disclosed modular unit(s) generally include (or communicate with) processing unit(s) that are adapted to run algorithm(s) that process such data to generate relevant metrics/measurements that address applicable use cases.

Thus, the noted information may be leveraged in various ways according to the present disclosure, e.g., providing real-time feedback to the prescribed user and his/her colleague(s) (e.g., parent(s)) and providing clinical feedback to the prescribing physician or health care provider, e.g., providing real-time or cumulatively collected information concerning brace usage and related anatomical parameters.

In exemplary implementations, the disclosed sensing and/or feedback mechanism includes force and/or positioning sensing functionality that may be associated with strap(s), webbing, ratchet(s), and/or other tensioning elements that are associated with orthotic and/or prosthetic devices. For example, the force and/or position sensing functionality may be associated with strap(s), webbing, ratchet(s), and/or other tensioning elements that are adapted to releasably fix an orthotic/prosthetic device in place. Thus, for example, a lower leg, ankle and foot orthotic brace may include one or more (e.g., three) straps for use in releasably fixing the brace relative to a prescribed user's ankle. At least one of the straps may be provided with a force sensor and/or a position sensor that is adapted to monitor and/or measure force or position, respectively. The sensor(s) may be advantageously integrated with the strap(s) (or other tensioning element, e.g., webbing or ratchet mechanism), although it is further contemplated that the sensor(s) may be detachably secured with respect thereto, e.g., using a conventional attachment mechanism such as a snap, a Velcro™ connection mechanism or the like.

The sensing/feedback mechanism may also advantageously include and/or interact with one or more communication functionalities that facilitate communication of the sensed parameters, e.g., force and/or position parameters. Exemplary communication functionalities include visual, haptic (vibratory) and/or auditory signals or cues. The foregoing signals/cues may be delivered in situ, i.e., directly from the modular unit that includes the sensing/feedback mechanism, or from a remote device, e.g., a smart/cellular phone, pager, personal digital assistant, tablet or the like. Thus, in exemplary embodiments of the present disclosure, the modular unit includes a communication capability, e.g., a short-range wireless communication transmitter that is Bluetooth compliant, that is adapted to transmit sensed/measured parameters to a remote device, e.g., a smart/cellular phone, computer or other electronic device, for processing, display and/or storage.

In exemplary embodiments, the disclosed position sensors integrated with the strap(s) of a brace are configured to measure the distance to which the strap has been pulled using inductive sensors. Thus, for example, at least one of the sensors may be an inductive sensor with coil technology and conductive material may be embedded in the strap(s). The inductive sensor(s) may advantageously interact with the conductive material producing a signal indicating the location of the inductive sensor with respect to the strap(s). In exemplary embodiments, the position sensor may include multiple coils and multiple conductive materials may be imbedded in the strap. In further exemplary embodiments, the conductive material may be shaped so as to define a variable width along the X-axis (as defined by the strap). In exemplary embodiments, the inductive sensor may be a LDC1312 unit that is commercially available from Texas Instruments (Dallas, Tex.).

In other embodiments, the inductive sensor may be used to measure the distance from the coil and a conductive material that interacts with a section of the orthotic or prosthetic device along the z-axis. The inductive sensor can sense the conductive material along the “z axis” allowing the inductive sensor to sense presence and determine the distance of the conductive material from the surface of the circuit board where the coil is located. The disclosed system and method may advantageously include and/or interact with data processing and/or analytical functionalities. Thus, the force and/or position parameters that are sensed/measured by the disclosed sensing/feedback mechanism(s) may be transmitted to a remote device (either directly or by way of an associated network) that is programmed to store, process, analyze and/or display the sensed/measured data. Various analytical tools may be supported by and/or incorporated into the disclosed systems and methods, e.g., analytics related to anatomical developments of the user, analytics related to usage frequency/duration, analytics related to force delivery, analytics related to suitability of an associated orthotic/prosthetic device in view of user growth/development/condition, and the like. The analytical results may be accessed by the prescribed user, by colleague(s) of the user (e.g., parents), and/or by the physician or health care provider(s). Historical information may be generated that may prove useful in longer-term treatment, recovery, conditioning and/or activities of the user and/or in developing a better clinical understanding of various treatment modalities and/or activity levels.

The disclosed systems and methods may be developed and delivered in conjunction with newly manufactured orthotic and/or prosthetic devices. In addition, the present disclosure contemplates retro-fitted applications of the disclosed modular units, e.g., through integration and/or association with existing or replacement straps, webbing, ratchet(s), and other tensioning mechanisms for use with existing orthotic and/or prosthetic devices. Still further, the modularity of the disclosed systems/methods permit flexibility in deployment and use of the underlying sensing/feedback mechanisms across a broad range of utilities and applications, i.e., a range of orthotic and prosthetic applications as well as other devices that may be worn and/or used by individuals, e.g., training apparatus, research devices and the like. Thus, the present disclosure provides efficient and cost-effective modular units that facilitate immediate and widespread adoption and use of the disclosed systems and methods, including adoption and/or integration at various stages of the existing supply chain for orthotic/prosthetic devices and other products/devices.

Additional features, functions and benefits associated with the disclosed systems and methods will become apparent from the detailed description which follows, particularly when read in conjunction with the appended figures.

### BRIEF DESCRIPTION OF FIGURES

To assist those of skill in the art in making, using and practicing the systems and methods disclosed herein, reference is made to the accompanying figures, wherein:

FIGS. 1A-C are top views of an exemplary strap and sensing assembly to be used with orthotic or prosthetic devices, according to the present disclosure;

FIG. 2 is a side view (partially in section) of an exemplary implementation of the disclosed strap and sensing assembly with respect to a brace according to the present disclosure;

FIG. 3 is an exploded view of strap and sensing assembly according to the present disclosure; FIG. 4A shows an orthogonal view of another possible configuration of the device with a moving bar to hold the strap in place according to the present disclosure;

FIG. 4B shows an orthogonal view of another possible configuration of the device with a rigid bar to hold the strap in place insertion of an upper leg stump of a person without a natural lower leg according to the present disclosure;

FIG. 5A depicts a strap assembly that includes a “magnetic-based” sensing system in two cinching positions;

FIG. 5B depicts a strap assembly that includes an inductive sensor based sensing system in to measure the tightness of a strap according to the present disclosure;

FIG. 5C depicts a strap assembly that includes a “resistance-based” sensing system;

FIGS. 6A-6D depict an embodiment of the strap assembly using inductive sensors to measure the position of a strap for a orthotic or prosthetic device according to the present disclosure;

FIGS. 7A-7D depict an embodiment of the strap assembly using multiple conductive materials and multiple coils according to the present disclosure;

FIG. 8A illustrates an exemplary implementation of a modular sensing system 800 according to the present disclosure;

FIG. 8B illustrates exemplary of the modular sensing system 800 mounted to two different modular head according to the present disclosure;

FIGS. 8C-8M illustrate different possible types of modular attachments according to the present disclosure;

FIG. 9A illustrates a rear portion of an alternative scoliosis brace is shown secured to the torso of a user according to the present disclosure;

FIG. 9B illustrates a rear portion of an alternative embodiment of the brace sensor mechanism is shown with a flexible lower back brace according to the present disclosure;

FIG. 9C illustrates a front portion of an alternative embodiment of the brace sensor mechanism is shown with a flexible upper back brace according to the present disclosure;

FIG. 9D illustrates side portion of an alternative embodiment of the brace sensor mechanism is shown with a knee brace according to the present disclosure;

FIG. 9E illustrates a front portion of an alternative embodiment of the brace sensor mechanism is shown an elbow brace with an arm sling secured to the torso of a user;

FIG. 9F illustrates a front portion of an exemplary orthotic foot brace is shown secured to a user according to the present disclosure;

FIG. 9G illustrates a prosthetic leg that includes an upper leg portion, a lower leg portion and a foot portion is shown with a cone-shaped cavity attached at the top of the upper leg portion according to the present disclosure;

FIG. 9H illustrates a front view of an alternative embodiment of the brace sensor mechanism shown with a prosthetic arm secured to the torso of a user;

FIG. 10 provides a schematic flowchart of exemplary data flow within the orthotic or prosthetic device in clinical calibration of the device and daily use of the device according to implementations of the present disclosure;

FIG. 11 provides a schematic flowchart of exemplary data flow according to implementations of the present disclosure;

FIG. 12A depicts an exemplary screenshots and of to mobile application showing long-term feedback via bar graphs and a prescription display according to the present disclosure;

FIG. 12B depicts a further exemplary screenshot that shows a web application for clinicians showing long-term data display and summaries, as well as prescription according to the present disclosure;

FIG. 13 provides an exemplary flowchart that illustrates a sequence of steps by which the disclosed system/method may be determine quality and compliance of the orthotic or prosthetic device use according to the present disclosure;

### DESCRIPTION OF EXEMPLARY EMBODIMENT(S)

According to the present disclosure, systems and methods are provided for monitoring and/or measuring parameters associated with the use of various devices/systems, e.g., orthotic devices and prosthetic devices, such as leg braces, scoliosis braces, arm slings, post-operative back braces, knee braces, prosthetic units and the like. In exemplary implementations, the disclosed systems and methods are adapted to communicate the monitored and/or measured parameters, e.g., through visual, haptic (vibratory) and/or auditory signals or cues. Moreover, the monitored and/or measured parameters may be transmitted to a remote device that is programmed to store, process, analyze and/or display the data. Various analytical tools may be supported by and/or incorporated in the disclosed systems and methods, e.g., analytics related to anatomical developments of the user, analytics related to usage frequency/duration, analytics related to force delivery, analytics related to suitability of an associated orthotic/prosthetic device in view of user growth/development/condition, and the like. The analytical results may be accessed by the prescribed user, by colleague(s) of the user (e.g., parents), and/or by the physician or health care provider(s).

Among the analytics supported by the modular units of the present disclosure, compliance of orthotic or prosthetic wear may be determined based on sensed/measured parameters according to the present disclosure and compliance information is typically used in the medical literature and in practice by physicians and other health care providers to describe the amount of time a patient wears a brace as compared to the amount of time the doctor prescribes the brace to be worn. For example, if a doctor prescribes that a brace be worn twenty three (23) hours per day, but the patient only wears the brace for twelve (12) hours per day, the patient would be deemed to be fifty two percent (52%) compliant with respect to brace wear. Among other analytics supported by the modular units of the present disclosure, the quality of orthotic or prosthetic wear may be determined based on sensed/measured parameters and is distinct from compliance. For purposes of the present disclosure, quality is a measure of how well a device (e.g., an orthotic or prosthetic device) is being worn. Quality of wear is distinguishable from compliance of wear because the device may not be tightened completely when the patient/user is wearing it. In such circumstance, the patient/user may be deemed “compliant” because the device is being worn, but the “quality” of wear is less than desirable.

The present disclosure advantageously provides systems and methods that allow the capture of metrics that may be used to evaluate a range of activities and performance parameters, e.g., compliance of device use, quality of device use, step count, user activity level, range of motion, device/user orientation, and other device- and user-related measurements. For example, the quality of wear may be determined by strap tension and/or strap position, as described herein.

Of note, strap position is currently used by doctors to give patients a guide to where to tighten a brace to each day. Since the ability to reach that position can change over time (e.g., due to weight gain, eating, etc.), a better measure of quality may be achieved according to the present disclosure based on the tension of the strap, or some combination of both tension or position. Of note, the strap position is currently used by doctors to give patients a guide to where to tighten the brace to each day. For example, the tightness of the straps on a scoliosis brace needs to be adjusted based on the prescription of the physician or health care provider. For example, the position to which the strap is pulled generally needs to be changed over a period of time for proper treatment. The disclosed systems/methods are advantageously able to detect both the compliance and quality of device wear, and adapt the metrics over time as determined by the physician.

Furthermore, the disclosed systems/methods are advantageously able to measure the distance between two points on a brace and determine the distance that a strap has been pulled without physician and health care provider assistance.

Indeed, methods for measuring parameters-of-interest may vary and/or evolve according to the present disclosure. The modular sensing devices described in the embodiments may also include one, or some combination of, sensors that are capable of measuring various parameters, such as force, excursion, acceleration, angular position, pressure, temperature, humidity and light. The raw value measurements provided by these sensing devices can be used to generate corresponding metrics related to wear time of an orthotic or prosthetic device, range of motion, activity (e.g., steps, speed, movement, running vs. walking), and the like. Moreover, an algorithm developed to measure compliance/quality or other parameters may be static or varied from time-to-time. For example, it may be desirable for an algorithm that is intended to measure compliance/quality to utilize different parameters and/or different target performance levels from time-to-time, e.g., based on the length of time that a user has been engaged in use of the relevant device.

Of note, the present disclosure provides systems and methods that enable measurement and communication of relevant parameters, as well as updates, refinements and/or variations in prescriptive parameters and/or targets for device use, e.g., based on determinations by health care professional(s) in view of reported measurements. Thus, the disclosed systems and methods permit health care professionals to update “prescriptions” at any time and from remote locations. For example, a health care professional is able to receive and evaluate compliance and quality of use (and/or other parameter(s)) in his/her office, and then to refine the relevant prescription so as to enhance and/or optimize device usage based on his/her professional judgment. Moreover, the disclosed systems and methods support and enable algorithmic-based updates, refinements and/or variations in parameters and/or targets for device use, e.g., based on comparisons of device-based performance parameters and target performance levels which algorithmically translate to updated, refined and/or varied device-based usage parameters. The disclosed feedback systems and methods may be modular in design, but sensed/measured data may be user-specific, i.e., communications associated with updated, refined and/or varied usage parameters are generally specific to an individual use case, and are generally communicated by conventional communication protocols, e.g., Bluetooth communications or the like. Before describing exemplary implementations with reference to the accompanying figures, the following outline of features/functions is provided by way of overview:

### Problems Addressed

1. Care providers have no way to make informed decisions on patient wear characteristics (defined below) in order to improve treatment. For example, orthotists and prosthetists often rely on patient reported data to adjust fit of braces and prosthetics, to change type of device, etc. Health insurance reimbursement is often based on the patient's level of activity or adherence to prescription regimens.

2. Patients often find it hard to put on orthotics or prosthetics correctly, in part because the devices often rely on the patients to tighten or otherwise don and doff the device. This can result in an uncomfortable fit and/or ineffective treatment.

3. Patients who wear orthotics and prosthetics often have no way to keep track of and set goals for activity, steps, range of motion, etc.

4. There currently exists a gap between low costs orthotics and prosthetics, which typically are purely mechanical devices with no electronics or sensing capabilities, and high end, expensive orthotics and prosthetics, which can provide a wealth of valuable information and feedback to users and care providers.

5. Companies who would like to integrate sensing/feedback capabilities into their existing orthotic and prosthetic devices must often start from scratch, and are unable to utilize existing work in the field.

### System Description

1. Feedback and sensing module is provided that is integrated or in line with a strap or webbing material, ratchet system or other tensioning element that is typically under tension for use in orthotics and prosthetics.

2. Device/module has onboard processing and feedback capabilities.

3. Device/module includes some combination of the following sensors
a. Force Sensor (Load Cell using Strain Gauges Configured in Whetstone Bridge)
b. Excursion Sensor (Measure distance between two points, potentially by measuring magnetic fields or by using inductive sensor coil and conductive target)
c. Accelerometer
d. Magnetometer
e. Gyroscope
f. Pressure Sensor
g. Temperature
h. Humidity
i. Light

4. Device can uses the above raw sensor values to gather metrics related to:
a. Wear time of orthotic/prosthetic
b. Range of motion of device and/or user's body parts
c. Activity (Steps, speed, movement, running vs. walking)
d. Tightness (tensile force) present in strap or webbing of orthotic/prosthetic
e. Distance between two points on orthotic/prosthetic device and or user's body
f. Orientation of orthotic/prosthetic device

5. Raw sensor values collected by device will be transformed into metrics of importance to patients and care providers by a series of custom algorithms developed for each orthotic/prosthetic application.
a. In one implementation, the raw sensor data will be sent (wirelessly) to a database and/or mobile device, where custom algorithms for each orthotic/prosthetic application will transform raw values to metrics of importance
b. This is important because the electronics and data collected by the device will be largely the same for a wide range of orthotics and prosthetics.
c. What will vary based on the application is how the raw data is analyzed on the web.
d. This is advantageous to developing custom hardware and sensing capabilities for each medical condition, as much of the core development work and costs can be saved.
e. The frequency with which the sensor readings occur can be varied across applications to optimize for battery life and memory capacity based upon the frequency of the individual sensors needed to determine the metrics of interest for that given application.

6. Device can deliver real time feedback to users
a. On board vibration, light or sound from device
b. Live stream data from device wirelessly to smartphone or computer, which will then give feedback to users

7. Device has two modes of operation
a. Constant, low power mode
   i. All day gathering of certain data (for example, steps)
   ii. Power down the sensors needed for feedback donning and doffing device
b. Feedback mode
   i. Activated by user (button press) or automatically selected based upon sensor readings (device detects it is being donned or doffed)
   ii. Selectively power on certain sensors/feedback mechanisms
   iii. Activated when patient is donning/doffing orthotic/prosthetic device

8. The sleep cycle parameters (frequency of sleep and length of sleep) can be varied wirelessly (via Bluetooth) to change the average power consumption and recording frequency of the system. For example in the scoliosis implementation, the device sleeps for 6 minutes in between sensor readings, but to measure activity in a prosthetic, the device can sleep for 15 milliseconds in between sensor readings.

9. Certain electronic systems can be powered on or off based on the relevant sensor readings of interest. This can be changed wirelessly (via Bluetooth) to optimize the battery life of the system.

10. Device can be used with modular attachment mechanisms to integrate with wide range of orthotics and prosthetics. The electronics required for many applications remains unchanged.

11. Device can deliver long-term feedback to users, by keeping track of goals and incentives.

12. Battery powered

13. Wireless connectivity (Bluetooth, Zigbee, Wi-Fi)

Exemplary implementations of the disclosed systems and methods are described herein. However, it is to be understood that the present disclosure is not limited by or to such exemplary implementations.

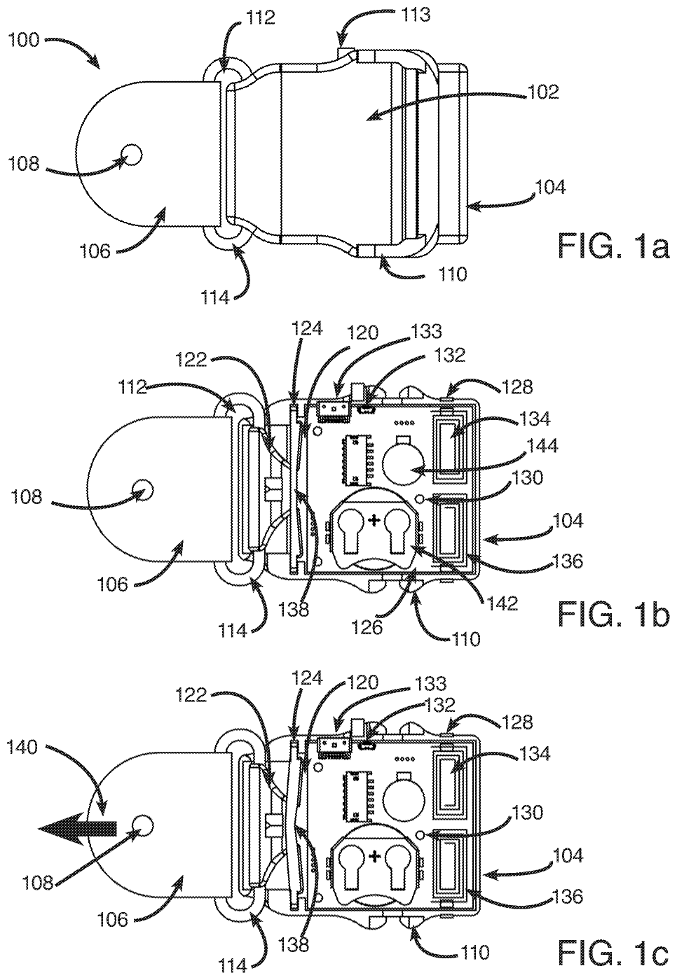

With reference to FIGS. 1A-C, top views of an exemplary strap and sensing assembly 100 to be used with orthotic or prosthetic devices, according to the present disclosure, are provided. In FIG. 1A, the top view of the sensing assembly 104 is covered by a top face 102. Strap and sensing assembly 100 includes a chafe 106 that includes an aperture 108 for use in securing the chafe 106 relative to a brace, and a sensing assembly 104 that is movably mounted relative to chafe 106. As noted above, chafe 106 may be secured relative to a brace using various mounting systems, e.g., a rivet or the like. In terms of brace-based mounting of chafe 106, it is noted that alternative mounting techniques may eliminate the need for aperture 108, as will be readily apparent to persons skilled in the art.

The chafe 106 can be mounted to the sensing assembly 104 using a mounting passage 114. The mounting passage 114 may pass through a connector (not shown in FIG. 1A) within the housing 110 and through an opening 116 in the chafe 106. In some embodiments the mounting passage can be a ring locked in a 90 degree position to the opening 116 of the chafe 106. The mounting passage 114 may form an elliptical shape forming a slot or passage 112 configured and dimensioned to receive a strap for use with braces. Alternative structural arrangements may be employed to define a slot or passage relative to housing 110, as will be readily apparent to persons skilled in the art.

The sensing assembly 104 includes a housing 110 and a gauge mechanism positioned (not shown in FIG. 1A) within the housing 110 that is adapted to measure the force applied to assembly 100. A switch or button 113 typically extends through or is otherwise associated with housing 110 of sensing assembly 104 to facilitate powering up or powering down of sensing assembly 104. Switch or button 113 interacts with electronics within housing 110, as described herein.

Turning to FIG. 1B, an interior of view of the sensing assembly 104 showing the interior components according to the present disclosure is provided. As shown therein, strap and sensing assembly 100 includes housing 110 that defines a cavity 120 for receipt of operative components of the disclosed sensing system. As previously noted, the mounting passage 114 defines a slot or passage 112 that is configured and dimensioned to receive a strap for use with braces. In the exemplary implementation of assembly 100, passage 112 is defined by the elliptical shape formed by the mounting passage 114 while connecting the strap 106 and the connector 122 within the housing 110 of the sensing assembly 104. The size and geometry of passage 112 is selected so as to permit ease of passage of a strap associated with the disclosed system. Alternative structural arrangements may be employed to define a slot or passage relative to housing 110, as will be readily apparent to persons skilled in the art.

The housing 110 of the sensing assembly 104 includes a connector 122 and a gauge mechanism 124 secured in a slot 138 of the connector 122. The connector 122 includes an opening for the passage of the mounting passage 114. The connector 122 extends perpendicular to the mounting passage 114 and on the opposite side of the mounting passage 114 the gauge mechanism 124 is secured to the connector in a slot 138 on the connector 122. The gauge mechanism 124 extends from a first end to a second end of the housing 110, parallel to the mounting passage 114.

The housing 110 of the sensing assembly 104 includes a circuit board 126 positioned within the cavity 120. The circuit board 126 is powered by a battery 142, which is also positioned within cavity 120 and which is in electrical communication with circuit board 126. Battery 142 provides power to the various elements of sensing assembly 104, as described herein.

The circuit board 126 may further communicate with one or more LEDs 128 that may be powered to provide data communication to users, caregivers and/or other healthcare providers. In instances where one or more LEDs 128 are included, the housing 110 generally includes one or more openings or windows to allow observation thereof. Circuit board 126 may also communicate with a speaker 130 that, when powered, is adapted to provide an aural signal as to performance of the brace system to users, caregivers and/or other healthcare providers. In instances where a speaker 130 is included, housing 110 generally include an opening to allow unobstructed passage of sound there through. Thus, the disclosed systems and methods of the present disclosure may be adapted to provide one or more forms of communication as to users, caregivers and/or other healthcare providers, e.g., visually observable communication (e.g., LEDs 128), aural communication (e.g., speaker 130), and/or tactile communication (e.g., vibratory motor 144).

A switch or button 113 is associated with housing 110 to allow users to power up/power down the disclosed sensing system. The switch/button 113 communicates with an associated electronic component 132 that is in electronic communication with circuit board 126 and translates the user interaction to the electronics of the system. The circuit board 126 may also include a USB port 133 that permits porting of data/programming to and from the electronics system. USB port 133 is accessible through an opening (not shown in FIGS. 1A-C) defined in housing 110.

In exemplary embodiments of the present disclosure, the gauge mechanism 124 takes the form of a strain gauge 124 that is positioned within housing 110 and that is cooperatively mounted with respect to chafe 106 so as to measure forces experienced thereby. For example, with reference to FIG. 1C, force can be applied along the X-axis in direction 140 pulling the chafe 106 away from the strain gauge 124, causing the strain gauge 124 to deflect. The strain gauge 124 can measure the force and can communicate the force measurements to an input associated with circuit board 126. The circuit board 126 may include processing functionality that is adapted to process the force measurements delivered by strain gauge 124. The circuit board 126 is also generally associated with transmissive elements, e.g., transceiver elements that include antenna and other components associated with conventional data communications, so as to facilitate transmission and receipt of data associated with measurements and control inputs. In another embodiment of the present disclosure, the circuit board 126 sensing assembly 104 can include inductive coils 136 and 134. The inductive coils 136 and 134 are cooperatively mounted with respect to chafe 106 so as to measure distance and position between two points on the strap.

The inductive coils 136 and 134 communicates tightness measurements to an input associated with circuit board 126. The circuit board 126 may include processing functionality that is adapted to process the tightness measurements delivered by inductive coils 136 and 134. The circuit board 126 is also generally associated with transmissive elements, e.g., transceiver elements that include antenna and other components associated with conventional data communications, so as to facilitate transmission and receipt of data associated with measurements and control inputs.

Circuit board 126 may be in communication with one or more components that are adapted to signal users, caregivers and/or healthcare providers as to the condition and operation of the disclosed sensing system. For example, circuit board 126 (and battery 142) may be in electronic communication with a vibration motor 144 that is adapted to be energized in response to control signals received and/or generated by the circuit board 126. For example, if the brace associated with sensing assembly 104 is insufficiently cinched or otherwise in need of attention/adjustment, circuit board 126 may be programmed to energize vibration motor 144 so as to alert the user of the situation. The vibratory function of vibratory motor may involve a sustained vibratory operation, or pulsed/intermittent vibratory operation, or both depending on the programming of the circuit board.

As a non-limiting example, the strap and sensing assembly may include strain gauge functionality that functions to measure the force level experienced by a device, e.g., a prosthetic or orthotic device. Thus, two strain gauges may be provided. A beam may be associated with the strain gauges such that beam bending correlates with a linear force applied to or experienced by the device. The strain gauges may be positioned in the region of bending such that a Wheatstone bridge is established therebetween. The strain-based signal generated by the Wheatstone bridge may be compared to reference data to determine whether the strap force is within a prescribed range. Moreover, changes in the signal may be monitored to assess performance of an orthotic or prosthetic brace over time. The strain-based signal generated by the Wheatstone bridge may be fed to a differential instrumentation amplifier which may be adapted to amplify the signal, e.g., to a level that may be read by an analog-to-digital converter associated with a microcontroller, as described in greater detail below. As with the “cinching” measurements described above, the strain-based measurements may be stored in a database for use in various analytic and/or diagnostic functions, e.g., assessing the degree to which a device has been properly employed by a user. Alternative systems may be used to monitor and/or measure forces experienced by the device, as will be readily apparent to persons skilled in the art.

As noted above, the disclosed sensing assembly may support a plurality of indicating lights, e.g., LED's, that are adapted to provide a visual signal to users and other caregivers as to the status of a brace. The LED's may be aligned in corresponding rows, e.g., along the edges of the housing, and may be adapted to illuminate in different colors based on the orientation/alignment of the associated orthotic or prosthetic device. Thus, when the device is properly adjusted to a user, sensing mechanisms associated with the disclosed sensing assembly are adapted to recognize the proper orientation/alignment and to signal that information to the user, e.g., by illuminating one or more “green” LED's. Conversely, if the sensing mechanisms associated with the disclosed sensing assembly determine that the device is not properly oriented/aligned, a warning signal may be provided to the user and other caregivers, e.g., by illuminated one or more “red” LED's. In exemplary implementations, the disclosed assembly may be provided with green, yellow and red LED's to facilitate an indication of device compliance (e.g., with green LED illumination corresponding to strong compliance, red LED illumination corresponding to poor compliance, and yellow LED illumination corresponding to an intermediate level of compliance).

Beyond visual indicators, it is further contemplated that additional and/or alternative communication modalities may be implemented according to the present disclosure. For example, the disclosed sensing assembly may further (or alternatively) include haptic (e.g., vibratory) and/or auditory functionalities for communicating information concerning orthotic or prosthetic device usage. The disclosed sensing assembly may thus be adapted to deliver vibratory impulses to the user when the device is improperly positioned, such vibratory impulses varying in intensity and/or frequency as the positioning/alignment of the device is adjusted. Similarly, the disclosed sensing assembly may be adapted to deliver vibratory impulses to the user when the device is properly positioned, such vibratory impulses varying in intensity and/or frequency as the positioning/alignment of the device is adjusted. The disclosed sensing assembly may also include an aural transmitter that is adapted to transmit sound-based signals to the user based on device positioning and/or usage, with differing aural signals based on relative positioning of the device. The breadth and flexibility of the communication modalities that may be implemented according to the present disclosure will be readily apparent to persons skilled in the art in view of the present disclosure.

The sensing assemblies that are adapted to provide advantageous monitoring and feedback functionality according to the present disclosure may be incorporated into newly constructed and prescribed orthotic or prosthetic systems, retrofitted onto existing systems, and/or used in conjunction with a range of orthotic, prosthetic and other user-worn devices/systems. Indeed, although individual prosthetic devices, and to some degree orthotic devices, are custom fabricated for specific users, operative elements of these systems are relatively uniform and therefore well adapted for retroactive transition to the monitoring/feedback system of the present disclosure. Thus, the disclosed modular monitoring/feedback functionalities may be widely adapted at minimal expense to users and/or health care providers across a range of clinical/user applications.

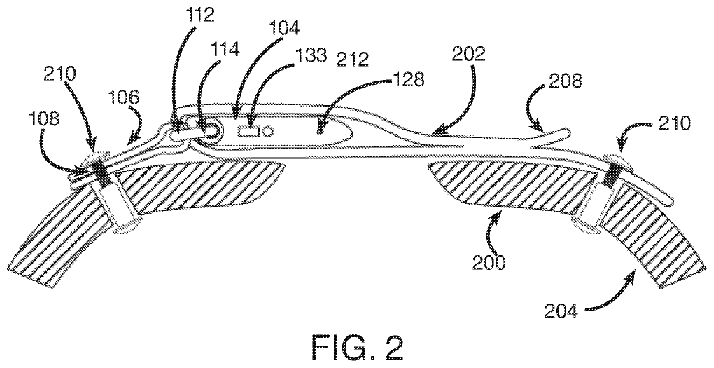

With reference to FIG. 2, a side view (partially in section) of an exemplary implementation of the disclosed strap and sensing assembly with respect to a brace. As shown therein, chafe 106 is mounted with respect to a brace 200 using a rivet 210 through aperture 108.

The sensing assembly 104 is connected to the chafe 106 using via a mounting passage 114. The sensing assembly includes 104 a USB port 133, LEDs 128, an aural communication device and/or and a tactile communication device. The mounting passage 114 may form an elliptical shape forming a slot or passage 112 configured and dimensioned to receive a strap 202 for use with braces. The strap 202 can connect a first end 204 of the brace 200 to the second end 206 of the brace 200. The strap 202 may be secured to the brace using a rivet 210 on the first end 204 of the brace 200. The strap 202 can pass under the sensing assembly 104 loop through the passage 112 and can pass over the sensing assembly 104. Once over the sensing assembly 104, the strap 202 is fixed, e.g., based on Velcro™ securement 208 relative to itself.

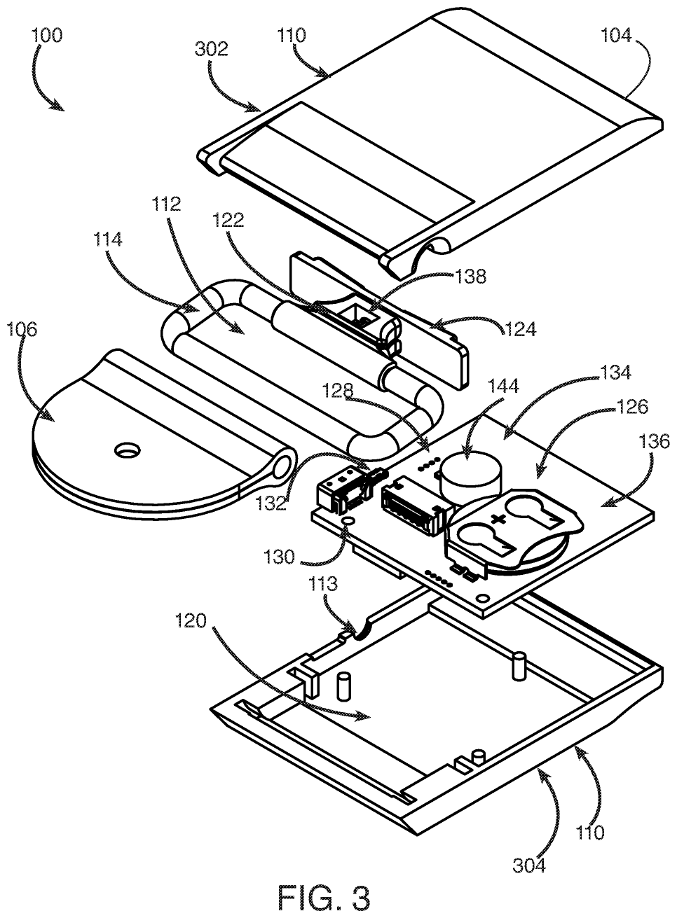

With reference to FIG. 3, an exploded view of strap and sensing assembly is provided. The strap and sensing assembly 100 includes a sensing assembly 104, mounting passage 114 and chafe 106. The sensing assembly 104 includes, a housing 110 and the housing 110. The housing includes a top portion 302 and a bottom portion 304. The top and bottom portion 302-304 of the housing 110 form a cavity 120. A connector 122, a strain gauge 124, and a circuit board 126 are disposed within the cavity 120. The mounting passage 114 connects the connector 122 to the chafe 106. The mounting passage passes through an opening of the connector 122 and passes through an opening 116 of the chafe 106. The mounting passage 114 forms an elliptical shape forming a passage 112 configured to receive a strap of a brace. The strain gauge 124 is secured to a slot 138 of the connector 122. The circuit board 126 may further communicate with one or more LEDs 128, speakers 130 and a vibratory motor 144 that may be powered to provide data communication to users, caregivers and/or other healthcare providers. The sensing assembly includes a switch/button 113 (as shown in FIGS. 1A-1C) communicates with an associated electronic component 132 that is in electronic communication with circuit board 126 and translates the user interaction to the electronics of the system.

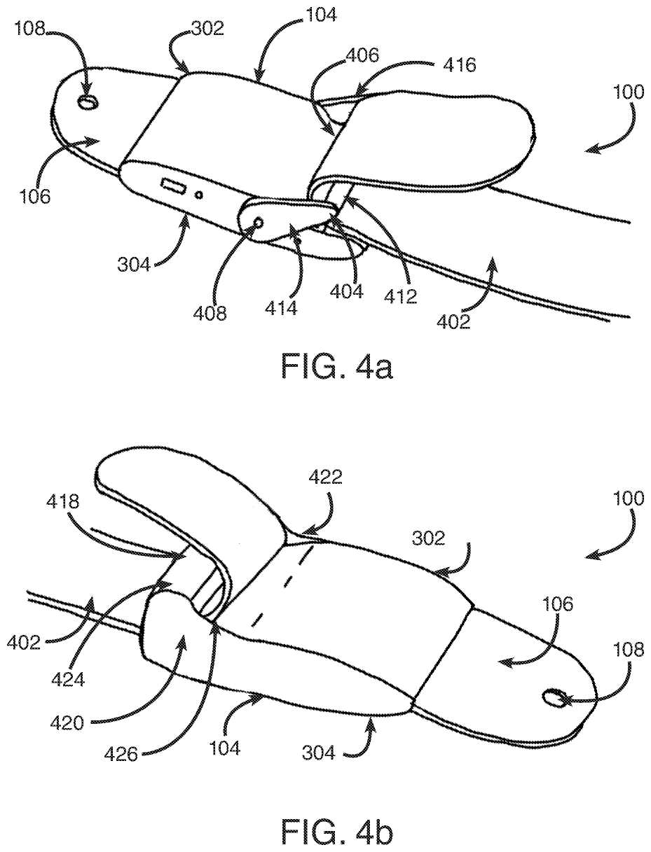

With reference to FIG. 4A, illustrates a mounting passage embodied as a moving bar in the strap and sensing assembly according to some embodiments. Modular sensor assemblies according to the present disclosure can be mounted to various types of orthotic and/or prosthetic devices in an in-line fashion, e.g., within a cinching loop-strap system. In some embodiments, the strap and sensing assembly 100 include a chafe 106, a sensing assembly 104, a strap 402, and a moving bar 404 as a mounting passage. The chafe 106 is secured to a first end of the sensing assembly 104. The moving bar 404 is secured to the second end of the sensing assembly 104. The moving bar 404 include two side walls 414 and 416 extending parallel to one another and connected by a bar 412 extending perpendicular from the two side walls 414 and 416. The two sidewalls 414 and 416 and the bar form an rectangular shape forming a slot or passage 406 configured and dimensioned to receive a strap 402 for use with braces. The moving bar 404 can be secured to the sensing assembly 104 at a hinge point 408 and 410 (not shown) disposed in between the top and bottom portion 302 and 304 of the housing 110 of the sensing assembly 104. The moving bar 404 can rotate circumferentially along an arc with a radius equal to the distance between the bar 412 and the top portion 302 of the housing of the sensing assembly 104. The moving bar 404 can be configured to receive one end of the strap 402 in between the bar 412 and the sensing assembly 104, through the passage 406 and loop over the bar 412. One end of the strap 402 may extend parallel to the other end of the strap 402 along a Z-axis. As will be readily apparent to those skilled in the art, the embodiment is not limited to a system with releasable loop assemblies on both sides of the sensing assembly 104.

With reference to FIG. 4B illustrates a mounting passage embodied as a top bar in the strap and sensing assembly according to some embodiments. In some embodiments, the strap and sensing assembly 100 include a chafe 106, a sensing assembly 104, a strap 402, and a top bar 418. The chafe 106 is secured to a first end of the sensing assembly 104. The top bar 418 is secured to the top portion 302 of the sensing assembly at a second end of the sensing assembly 104. The top bar 418 includes two side walls 420 and 422 extending parallel to one another and connected by a bar 424 extending perpendicular from the two side walls 420 and 422. The two side walls 420-422 can be secured to the top portion 302 of the sensing assembly 104. The bar 424 and the two side walls 420-422 can form a passage 426 configured to receive one end of the strap 402 in between the bar 424 and the sensing assembly 104, through the passage 426 and loop over the bar 424. One end of the strap 402 may extend parallel to the other end of the strap 402 along a Z-axis. As will be readily apparent to those skilled in the art, the embodiment is not limited to a system with releasable loop assemblies on both sides of the sensing assembly 104.

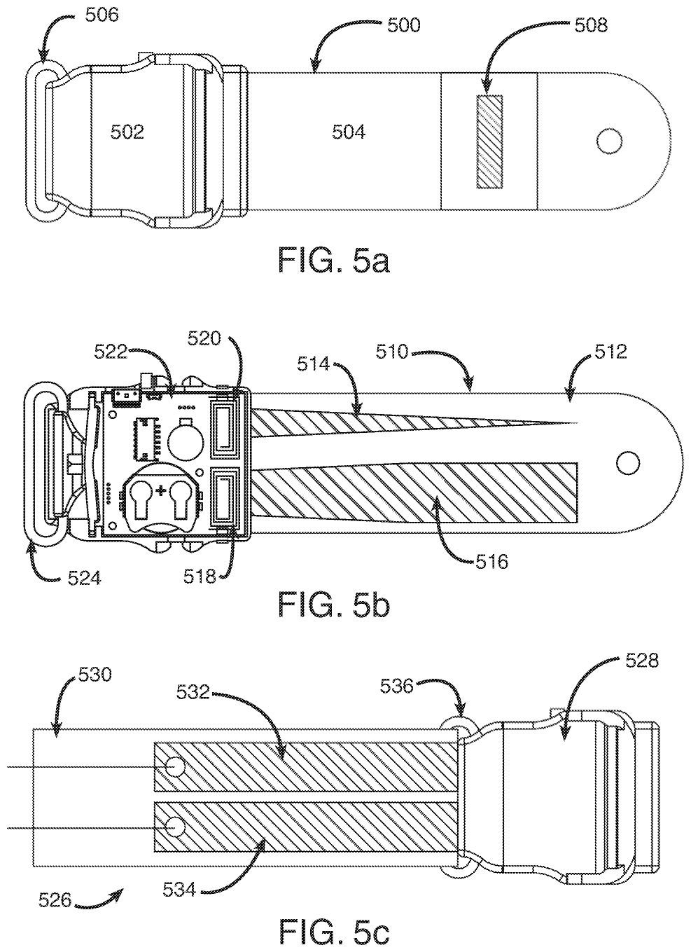

With reference to FIGS. 5A-5C, top views of exemplary implementations of the disclosed modular sensing assembly in conjunction with orthotic or prosthetic devices are provided. FIG. 5A depicts a strap assembly that includes a “magnetic-based” sensing system in two cinching positions. FIG. 5B depicts a strap assembly that includes an inductive sensor based sensing system in to measure the tightness of a strap. FIG. 5C depicts a strap assembly that includes a “resistance-based” sensing system. Each of the disclosed sensing systems is adapted to monitor/measure the position of the strap, e.g., when used to cinch a brace/device around the body part of a user. The sensing parameter may be compared to a target reading to determine whether the brace/device is properly tightened (subject to applicable tolerances). Based on such comparison, a signal may be delivered to the user and associated caregivers (e.g., a visual, haptic and/or aural signal). Moreover, the determination may be stored in a database for use in various analytic and/or diagnostic functions, e.g., assessing the degree to which a brace has been properly employed by a user.

Turning to FIG. 5A, exemplary strap assembly 500 that includes a sensing assembly 502 that is mounted with respect to a strap 504 facilitates cinching of a brace (not pictured). A mounting passage 506 is secured to the sensing assembly 502 through a connector (not pictured).

Magnetic sensors are embedded or otherwise associated with sensing assembly 502 (not pictured) and are configured/positioned so that as the output voltage of one magnetic sensor associated with sensing assembly 502 increases and the output voltage of the second magnetic sensor associated with the sensing assembly 502 decreases as the magnet 508—which is embedded or otherwise associated with a region toward or at the other end of the strap 504—moves relative to sensing assembly 252. Thus, the difference between the two output voltages generated by the magnet sensors associated with sensing assembly 502 increases as magnet 508 moves closer to the sensing assembly 502, i.e., the difference in output voltage measured by the disclosed system will vary based on the position of strap member 504 relative to sensing assembly 502, i.e., the degree to which strap member 504 is “cinched” in securing the orthotic or prosthetic brace relative to a user's body part. As with the resistance-based implementation described above, the signal generated by the disclosed output voltage measurement may be amplified and transmitted to an analog-to-digital converter associated with a microcontroller.

Turning to FIG. 5B, exemplary strap assembly 510 includes a sensing assembly 522 that is mounted with respect to strap 512 which facilitates clinching of the brace (not pictured). First and second conductive strips 514, 516 are embedded or otherwise associated with the strap 512. The strap 512 can be configured to pass over the sensing assembly 522 through the mounting passage 524. The conductive strips 514, 516 can be of variable width. The sensing assembly 522 includes a circuit board 520. The circuit board 520 includes inductive sensors including coils 518, 520. The sensing assembly 522 generates a signal based on an interaction between the coils of the inductive sensors 518, 520 as the conductive fabrics 514, 516 pass over or under the sensing assembly 522. The signal changes as the width of the conductive strips 514, 516 increases or decreases. The positioning/tightness of the strap 512 can be advantageously determined according to the present disclosure based on interaction between the conductive strips 514, 516 embedded in or applied to the strap 512 and the inductive sensor in the sensing assembly 522. In exemplary embodiments, the inductive sensor can measure the distance between a first point on a brace (not shown) and a second point on the opposite side of the brace (not shown), as described in greater detail below.

Turning to FIG. 5C, exemplary strap assembly 526 includes a sensing assembly 528 that is mounted with respect to a strap 530 which facilitates cinching of a brace (not pictured). First and second resistive fabric strips 532, 534 are embedded or otherwise associated with strap 530. The fabric-based circuit acts as a custom, flexible linear potentiometer. An electronics module is incorporated into or otherwise associated with sensing assembly 528. The electronics module is adapted to amplify the signal generated based on a resistance change between opposed points along resistive strips 532, 534. The mounting passage 536 around which strap 530 passes is also conductive and bridges resistive strips 532, 534 when the strap assembly 510 is used to secure a brace relative to a user. As the conductive mounting passage 536 bridges the two resistive strips 532, 534 the resistance changes linearly. Indeed, the system functions as a Wheatstone bridge, generating a signal based on the relative positioning of the elements. Thus, the resistance measured by the disclosed system will vary based on the position of strap member 530 relative to conductive mounting passage 536, i.e., the degree to which strap member 530 is “cinched” in securing the orthotic or prosthetic brace relative to a user's body part. Of note, the signal generated by the disclosed resistance measurement may be amplified (e.g., using a Texas Instruments INA126 amplifier) and transmitted to an analog-to-digital converter associated with a microcontroller.

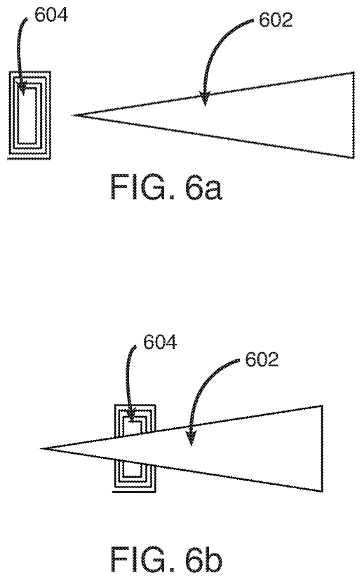

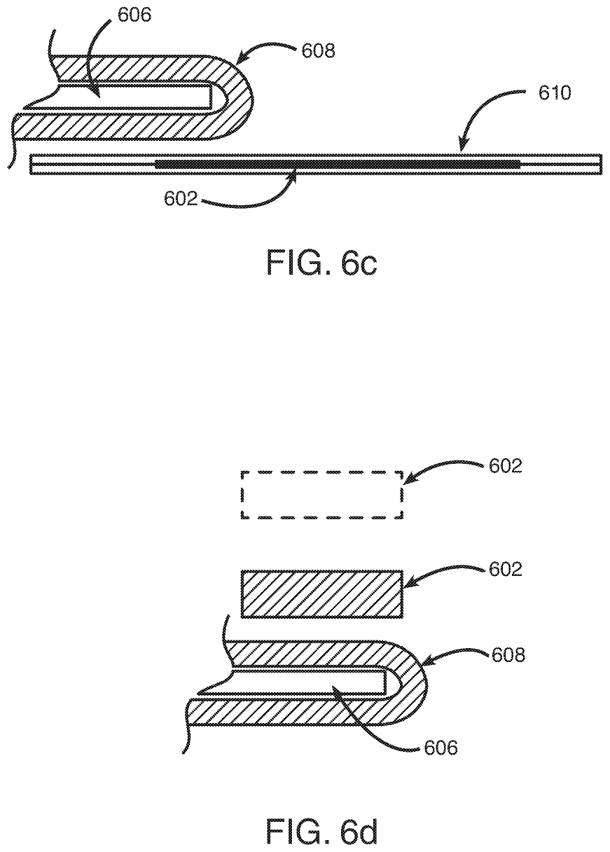

With reference to FIGS. 6A-6D depict an embodiment of the strap assembly using inductive sensors to measure the position of a strap for a orthotic or prosthetic device. Turning to FIGS. 6A-6B, in exemplary embodiments, the conductive material 602 may be embedded in and/or applied to a surface of the strap. A sensing assembly may be mounted with respect to the strap and may include an inductive sensor. The conductive material 602 may be for example, foil, fabric, copper, aluminum or platinum. In exemplary embodiments, the conductive material 602 is shaped with variable width along the X-axis. The positioning/tightness of the strap can be advantageously determined according to the present disclosure based on interaction between the conductive material 602 embedded and the inductive sensor. For example, the inductive sensor may include a coil 604 attached to or otherwise in communication with a printed circuit board. Turning to FIG. 6B the inductive sensor may produce an alternating current through the coil 602 as the conductive material 602 passes over the coil 604. The alternating current creates a first alternating magnetic field which induces an alternating electrical current (eddy current) in the conductive material 602. The eddy current in turn creates a second magnetic field that couples with the first magnetic field created by the coil 604. The inductive sensor measures the effect of a nearby conductor by measuring the frequency shift caused by the coupling of the first and second magnetic field. Turning to FIG. 6C, the coil 604 can be included in the circuit board 606 inside the sensing assembly 608. While the strap is in an initial position in clinical use, the inductive sensor may be disposed parallel to the strap along the X-axis. The sensing assembly 608 may interact with the conductive material 602 embedded in the strap 610 as the as the conductive material passes over the sensing assembly 608. In exemplary embodiments, the inductive sensor may produce a signal based on the measured frequency shift. In exemplary embodiments, due to the variable width of the conductive material 602 , a variable amount of area may be exposed to the first magnetic field created by the coil 604(as shown in FIGS. 6A-B). For example, with reference to FIG. 6B and 6C, as the strap 610 passes over the sensing assembly 608 along the X-axis, a wider area of the triangle shaped conductive material 602 is exposed to the first magnetic field. The effect of the amount of conductive material 602 exposed to the first magnetic field may be used to determine the distance between the conductive material 602 and the coil 604 along the X-axis, and accordingly what position the strap 610 has been pulled to along the X-axis.

Turning to FIG. 6D, the conductive material 602 can move along the Z-axis as shown by 602 and 602′. Consequently, the signal produced by the interaction of the sensing assembly 610 and the conductive material 602 may vary based on the distance along the Z-axis and the X-axis.

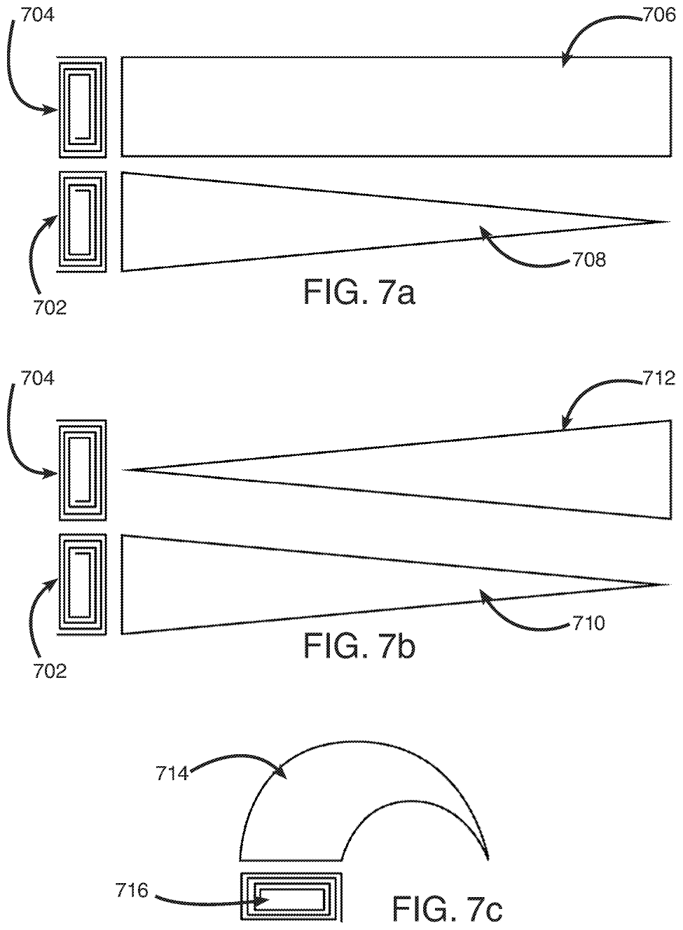

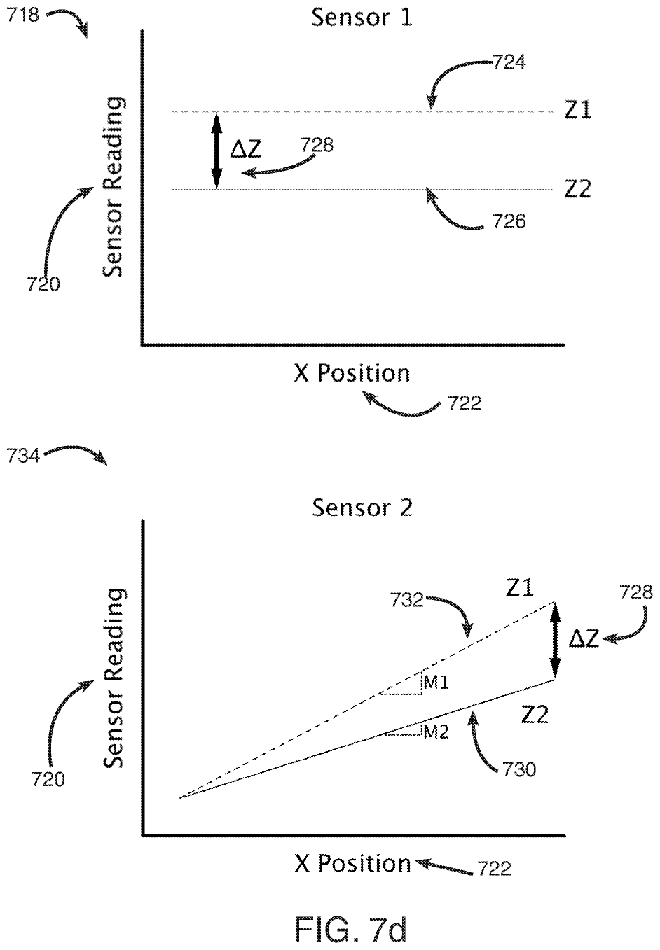

With reference to FIGS. 7A-7D depict an embodiment of the strap assembly using multiple conductive materials and multiple coils. As mentioned above, the conductive material can move along the X-axis and Z-axis. FIGS. 7A-B illustrate an exemplary two coil design for position sensing using inductive sensors according to exemplary embodiments of the present disclosure. Turning to FIG. 7A, in exemplary embodiments two coils 702 and 704 may be disposed in a parallel alignment along the X-axis relative to two conductive materials 706 and 708 disposed along the strap (not shown). In exemplary embodiments, in an initial position, conductive materials 706 and 708 may be disposed parallel to each other along the Y-axis. In addition, the coils 702 and 704 may be disposed parallel to each other along the Y-axis. In exemplary embodiments, the conductive material 708 may have variable width along the X-axis while conductive material 706 may have a constant width along the X-axis. For example, the conductive material 708 may be a triangle, spiral, circle, oval or another shape that has a variable width along the X-axis and the conductive material 706 may be a rectangle, square or another shape with constant width along the X-axis.

In exemplary embodiments, the inductive sensor within the sensing assembly (not shown) may generate signals that are used to calculate the distance between coil 702 and conductive material 708 and to calculate the distance between coil 704 and conductive material 706. Since the conductive material 706 has a shape with constant width along the X-axis, the signal produced by coupling of the magnetic fields produced by coil 704 and conductive material 706 does not vary along the X-axis and only varies along the Z-axis. Consequently, the inductive sensor may generate signals useful in determining the relative distance between coil 704 and conductive material 706 along the Z-axis. In exemplary embodiments, the disclosed system may use the distance calculations between coil 704 and conductive material 706 along the Z-axis to compensate/refine the distance calculations between coil 702 and 708 along the X-axis.

FIG. 7B illustrates an exemplary two coil design using two conductive materials with variable width along the X-axis according to the present disclosure. In exemplary embodiments, coils 702 and 704 may be disposed parallel along the X-axis relative to the conductive materials 710 and 712. In exemplary embodiments, in an initial position, conductive materials 710 and 712 may be disposed parallel to each other along the Y-axis. In addition, the coils 702 and 704 may be disposed parallel to each other along the Y-axis. The shapes of conductive materials 710 and 712 may both have variable width along the X-axis. In exemplary embodiments, the conductive materials 710 and 712 may be triangles.

In exemplary embodiments, the shape of conductive material 710 may be oriented in the opposite direction as compared to the shape orientation of conductive material 712. Consequently, as the width of conductive material 712 increases along the X-axis, the width of conductive material 210 decreases along the X-axis. In exemplary embodiments, as the inductive sensor in the sensing assembly passes over the conductive materials 710 and 712, the coils 702 and 704 will interact differently with the respective conductive materials. For example, the signal created by the magnetic field produced by the conductive material 710 while coupling with the magnetic field produced by the coil 704 will become weaker moving along the X-axis as the width of the conductive material gets smaller. Conversely, the magnetic field produced by conductive material 712 while coupling with the magnetic field produced by coil 702 will grow stronger moving along the X-axis as the width of the conductive material 712 increases. Consequently, the disclosed system may advantageously use the signals produced by the coupling of the magnetic fields of coil 704 and conductive material 712 to determine the distance between the coil 702 and conductive material 710 along the Z-axis. The disclosed system may also use the determined distance along the Z-axis to compensate/refine the distance measurement between the coil 702 and conductive material 710 along the X-axis. With reference to FIG. 7C the variable width of the conductive material 714 interacting with the coil 716 as the conductive material wraps around is depicted. As mentioned above the conductive material 714 is embedded or associated with a strap (not shown). Therefore, the conductive material 714 will wrap around along with the strap.

FIG. 7D provides graphs illustrating the Z-axis compensation process according to exemplary embodiments. In exemplary embodiments, graph 718 provides a depiction that illustrates distance measurements between the coil and conductive material having a constant width along the x-axis. The y-axis of graph represents the sensor reading 720, while the x-axis represents the x-position 722. The line 724 represents the sensor reading from conductive material 704 and coil 706 (as shown in FIG. 7A) moving along the x-axis in a first position on the z-axis. The line 726 sensor reading from conductive material 704 and coil 706 moving along the x-axis a second position on the z-axis. The graph 718 illustrates that the sensor reading 720 decreases from line 724 to line 726, therefore the change in the distance 728 along z-axis can be measured by taking the difference position of line 726 and with the position of line 724.

The graph 734 provides a depiction of the measurement of between the coil and conductive material with variable width along the x-axis. The y-axis of graph 734 represents the sensor reading 720, while the x-axis represents the x-position 722. The graph 730 measures the sensor reading 720 of the signal produced by the interaction of the coil 702 with the conductive material 708 (as shown in FIG. 7A). The line 730 indicates the sensor reading 720 as the conductive material interacts with the coil along the x-axis a first position on the z-axis. The line 732 indicates the sensor reading 720 as the conductive material interacts with the coil along the x-axis a second position on the z-axis. The sensor readings of lines 730 and 732 can be compensated by the determined difference in distance along the z-axis 728.

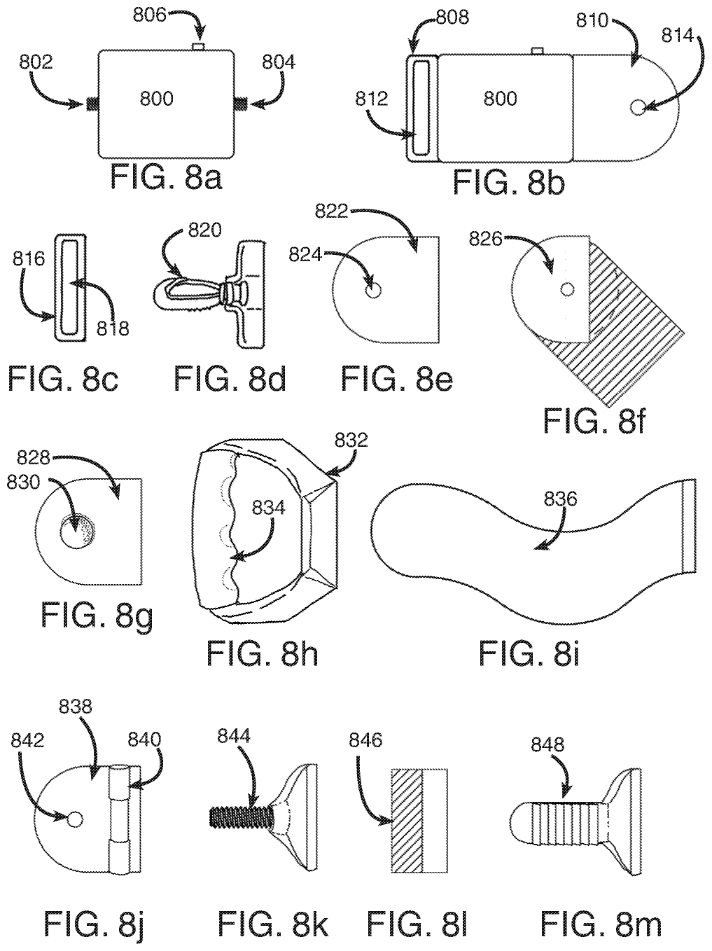

With reference to FIGS. 8A, an exemplary implementation of a modular sensing system 800 as described above is provided. The modular sensing system 800 includes a central electronics unit and attachment points such as clips 802, 804 to different modular heads (not shown in FIG. 8A). These clips 802, 804 can secure modular heads which can either be attached permanently or remain removable. The clips 802 and 804 at the bottom of modular sensing system 800 facilitate attachment of the modular sensing system 800 to a mounting device that permits attachment to an orthotic or prosthetic brace.

With reference to FIG. 8B, an exemplary of the modular sensing system 800 mounted to two different modular heads is provided. On a first end a mounting passage 812 is secured to the clip 802 of the modular sensing system 800. The mounting passage includes an aperture 812. The aperture 812, permits the modular sensing system 800 to be attached to a strap system (not shown) in a loop fashion. The loop strap can be releasably or fixedly attached to the modular sensing system 800 through aperture 812 on one end of the strap, while the other end of the strap is fixedly attached to a brace. On a second end of the modular sensing system 800 is a second modular end 810 is secured to the clip 804 of the modular sensing system 800. The modular end 810 includes an aperture 814. The modular end 810 may be secured relative to a brace using various mounting systems using aperture 814, e.g., a rivet or the like. In terms of brace-based mounting of modular end 810, it is noted that alternative mounting techniques may eliminate the need for aperture 814, as will be readily apparent to persons skilled in the art.

With reference to FIGS. 8C-8M, different possible types of modular attachments are provided. Turning to FIG. 8C, a mounting system 818 is provided including with a large rectangular aperture 818 that through which a strap can be threaded and looped back upon itself to releasably or fixedly attach. FIG. 8D shows an embodiment where the mounting system is secured through a hook/clasp 820 end that can easily be hooked or unhooked from a ring or loop. As will be apparent to those skilled in the art, an alternative embodiment of this mounting system is the connection of such a “hook mounting system”, in-line with the feedback sensor, to physical therapy or exercise equipment to monitor and provide feedback of tension in a strap system. FIG. 8E shows an embodiment where a mounting system is a fixed modular end 822 with a aperture 824. The fixed modular end 822 may be secured relative to a brace using various mounting systems using aperture 824, e.g., a rivet or the like. In some embodiments, the fixed modular end 822 can be rigid. In other embodiments, the fixed modular end 822 can be flexible. FIG. 8F shows an embodiment where a mounting system is a pivot end 826 that can rotate circumferentially. The pivot end 826 can cooperate with the modular sensing assembly and provide sensor information about angle or rotation. FIG. 8G shows an embodiment where a mounting system is a snap button end mounting system 828 including an aperture 830 that can be easily fixed and unfixed from a corresponding snap mounted to the brace. FIG. 8H shows an embodiment where a mounting system is a handle 832 that can be secured to the brace. The handle 832 can include grooves 834 configured for a user to grasp the handle 832 and pull the brace in a certain direction. FIG. 8I shows a strap end 836 that can be mounted on the brace. The strap end 836 can be made of Velcro® material or other types of webbing. The strap end 836 an interact with other loops or devices to tighten or loosen the brace. FIG. 8J shows an embodiment where a mounting system is an hinged end 838. The hinged end 838 can include an aperture 842 which can be used to secure itself to the brace using a rivet or the like. The hinged end 838 can also include a hinge 840 allowing the hinged end 838 to rotate about the hinge axis. FIG. 8K shows an embodiment where a mounting system is a threaded end 844 that can be screwed into a corresponding tapped hole. FIG. 8L shows an embodiment where a mounting system is a magnetic end 846 that can attach and be removed from a metal or additional magnetic attachment of a brace or modular sensing system. FIG. 8L shows an embodiment where a mounting system is a ratcheting end 848 that feeds into an additional ratchet mechanism that allows incremental tightening.

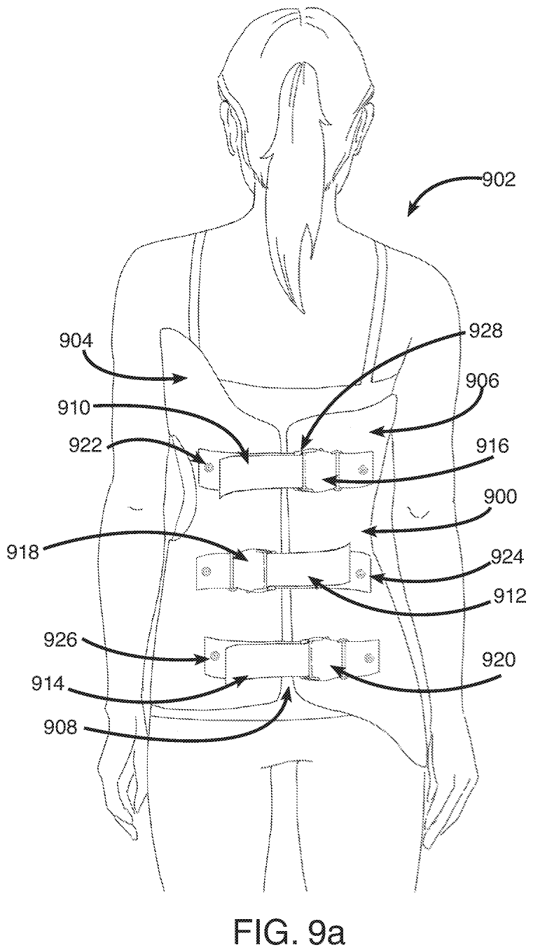

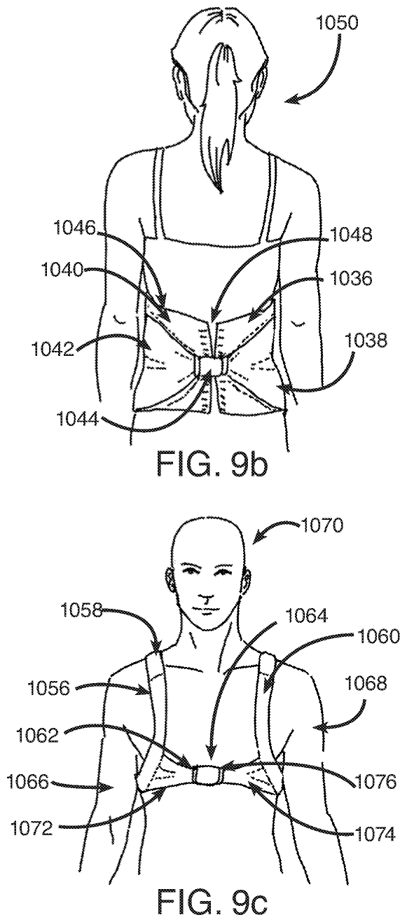

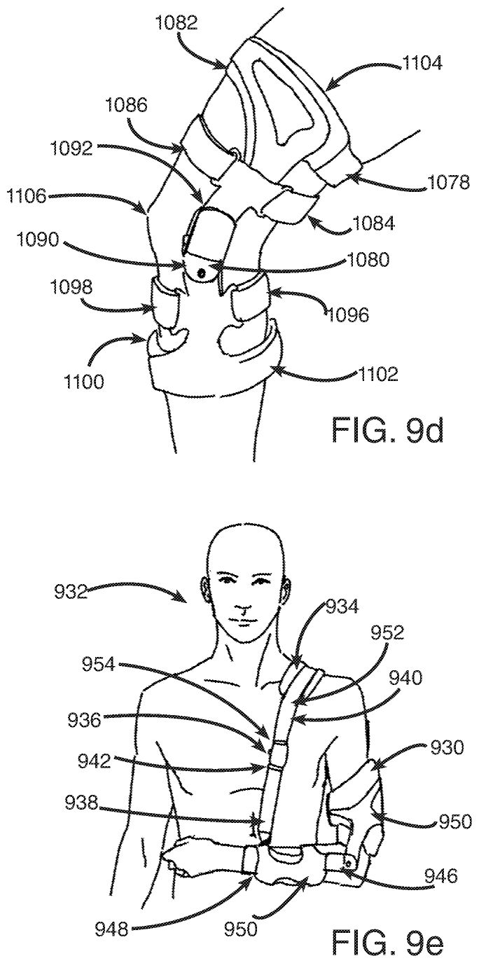

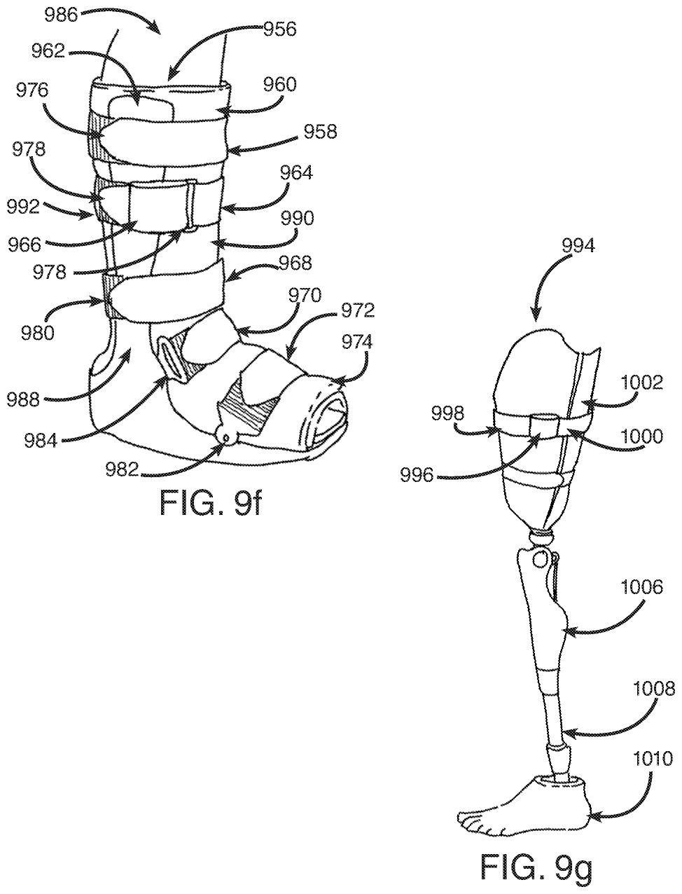

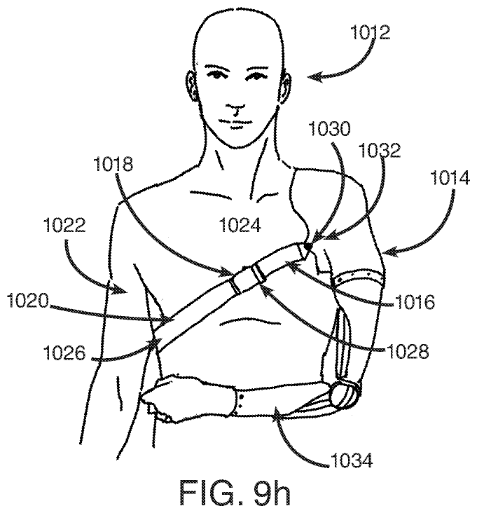

With reference to FIGS. 9A-9H, multiple embodiments, of modular sensor assemblies according to the present disclosure mounted to various types of orthotic and/or prosthetic devices in an in-line fashion, e.g., within a cinching loop-strap system are depicted. With initial reference to FIG. 9A, a rear portion of an alternative scoliosis brace 900 is shown secured to the torso of a user 902. The scoliosis brace 900 is cinched at the rear of user 902. Rear cinching braces are generally utilized for all-day wear, i.e., rear cinching braces are frequently prescribed for up to twenty three (23) hours of usage per day). Scoliosis brace 900 includes first and second portions 904, 906 that define a gap 908 therebetween. Of note, the “gap” defined by first and second portions 904, 906 may be a spacing therebetween or an overlap of first and second portions 904, 906. Thus, as noted above, the term “gap” as used herein should be understood to embrace the relative positioning of the first and second portions, whether such relative positioning defines spacing, overlap or even side-by-side juxtaposition.

A plurality of straps are mounted with respect to scoliosis brace 900 to facilitate securement thereof with respect to the user's torso. In particular, exemplary scoliosis brace 900 includes first strap 910, second strap 912 and third strap 914. As will be readily apparent to persons skilled in the art, the present disclosure is not limited to brace implementations that include three straps. Rather, the present disclosure may be implemented with fewer or greater numbers of straps without departing from the spirit or scope of the present disclosure.

With further reference to FIG. 9A, each of the straps is fixedly mounted with respect to either the first portion 904 or the second portion 906 of scoliosis brace 900. More particularly, first strap 910 is fixedly mounted with respect to first portion 904 by attachment element 922 and third strap 914 is fixedly mounted with respect to first portion 904 by attachment element 926. In the disclosed embodiment, second strap 912 is fixedly mounted with respect to second portion 906 by attachment element 924. Attachment elements 922, 924, 926 generally take the form of a rivet or like structure, thereby permitting rotational freedom so as to facilitate strap alignment in use.

Sensing assemblies 916, 918 and 918 are provided with respect to first, second and third straps 910, 912 and 914, respectively. Each of the sensing assemblies is mounted with respect to either first portion 904 or second portion 906 of scoliosis brace 900, e.g., by way of a mounting strap that is secured relative to the brace by a rivet or the like. In the exemplary embodiment two straps are fixed with respect to the first portion 904, whereas the intermediate strap is fixed with respect to the second portion 906. The alternating fixation arrangement of scoliosis brace 900 may improve the stability and/or ease with which the scoliosis brace may be brought into a desired orientation by the user, although the present disclosure is not limited by or to the disclosed alternating fixation arrangement.

The sensing assembly 906 includes a mounting passage 928 that accommodates passage of first strap 910 in a “looping” fashion, thereby allowing the user 902 to pull on the free end of strap 910 to cinch second portion 906 relative to first portion 904, thereby reducing the width of gap 908. Once cinched to a desired degree, strap 910 is generally adapted to be detachably fixed in the desired position, e.g., by way of cooperative Velcro™ interaction in the overlapping region of strap 910. Alternative fixation mechanisms may be employed to secure strap 910 in its cinched orientation, as will be readily apparent to persons skilled in the art. Looping, cinching and fixation mechanisms are generally provided with respect to second strap 912 and third strap 914, thereby permitting the user to bring the first portion 904 and the second portion 906 of scoliosis brace into a desired approximation.

With reference to FIG. 9B, a rear portion of an alternative embodiment of the brace sensor mechanism is shown with a flexible lower back brace 1046 is provided. The flexible lower back brace 1046 wraps around the abdominal area of the user 1050 and is secured to the user's lower back area. The flexible lower back brace 1046 includes a first and second portions 1040 and 1036 that define gap 1048 there between. Thus, as noted above, the term “gap” as used herein should be understood to embrace the relative positioning of the first and second portions, whether such relative positioning defines spacing, overlap or even side-by-side juxtaposition.

A first and second strap 1038, 1042 are mounted with respect to flexible lower back brace 1046 to facilitate securement thereof with respect to the user's lower back. As will be readily apparent to persons skilled in the art, the present disclosure is not limited to brace implementations that include two straps. Rather, the present disclosure may be implemented with fewer or greater numbers of straps without departing from the spirit or scope of the present disclosure. The first strap 1038 wraps around the abdominal area of the user 1050 to secure the first portion 1036 of the flexible lower back brace 1046 into place. The second strap 1042 wraps around the abdominal area of the user 1050 to secure the second portion 1040 of the flexible lower back brace 1046. The tightening of the straps 1038, 1042 reduces the size of the gap 1048 between the first and second portions 1036, 1040. Conversely, loosening of the straps 1038, 1042 widens the gap 1048 between the first and second portions 1036, 1040.

A sensing assembly 1044 is mounted with respect to first and second strap 1038, 1042 using a mounting passage 1052 and 1054 respectively to measure the tightness/compliance of the flexible lower back brace. The mounting passages 1052, 1054 accommodate passage of straps 1038, 1042 in a “looping” fashion. The straps 1038, 1042 to a desired degree, are generally adapted to be detachably fixed in the desired position, e.g., by way of cooperative Velcro™ interaction in the overlapping region of straps. Alternative fixation mechanisms may be employed to secure the straps 910 in its cinched orientation, as will be readily apparent to persons skilled in the art. With reference to FIG. 9C, a front portion of an alternative embodiment of the brace sensor mechanism is shown with a flexible upper back brace 1056 is provided. The flexible upper back brace 1056 can include a first and second strap 1058 and 1060, which wrap over the upper back area of the user 1070, and secure around each arm of the user 1066 and 1068. The flexible upper back brace 1056 can include a third strap and forth strap 1072, 1074 that wrap around the torso of the user 1070.

A sensing assembly 1064 can be mounted with respect to the third strap and forth strap 1072 and 1074. The third and fourth straps 1072, 1074 can tighten or loosen the flexible upper back brace. The sensing assembly can be configured to sense the tightness/compliance of the flexible upper back brace. The sensing assembly 1064 can be mounted to the third and fourth strap using mounting passages 1062, 1076. The mounting passages 1062, 1076 accommodate passage of straps 1072, 1074 in a “looping” fashion. The straps 1072, 1074 to a desired degree, are generally adapted to be detachably fixed in the desired position, e.g., by way of cooperative Velcro™ interaction in the overlapping region of straps.

With reference to FIG. 9D, a side portion of an alternative embodiment of the brace sensor mechanism is shown with a knee brace 1104 is provided. The knee brace 1104, is configured to provide stabilization and control of the user's 1106 knee joint. The knee brace 1104 includes an hinged upright 1080 secured to a first pad, second pad, third pad and forth pad 1082, 1086, 1098, 1100 respectively extending from the thigh area above the knee to the shin area below the knee. The first and second pad 1082, 1086 are located on the upper thigh area of the user 1106, above the knee. The third and fourth pad 1098, 1100 are located in the shin area below the knee. The knee brace 1104 can further include a first strap, second strap, third strap and forth strap 1078, 1084, 1096 and 1102. The straps can secure the respective pads to the hinged upright 1080. The hinged upright can provide controlled and stabilized movement to the knee joint of the user 1106.

A sensing assembly 1092 can be mounted to the hinged upright 1080 using a mounting assembly. The mounting assembly can be a hinged end 1090 and can include an aperture which can be used to secure itself to the hinged upright 1080 using a rivet or the like. The hinged end 1090 can also include a hinge allowing the hinged end 1090 to rotate about the hinge axis. In exemplary embodiments, the hinged axis will be limited to the range of the motion of the user's knee joint. The sensing assembly 1092 can be configured to measure range of motion of the knee.

With reference to FIG. 9E, a front portion of an alternative embodiment of the brace sensor mechanism is shown an elbow brace with an arm sling 934 secured to the torso of a user 932 is provided. The elbow brace 934 includes an arm sling 930, attached to a strap arrangement 952, which also relies on tension in the strap to facilitate immobilization of a limb, such as an arm in this embodiment. Strap 940 is fixedly attached at one end to the elbow area 946 of arm sling 930, e.g., by way of a rivet, Chicago binding post or the like. Strap 940 wraps around the back portion of the neck (not shown) of user 932, loops over the opposite shoulder to that of elbow area 946 and attaches to a sensor assembly 936 on the front side of the torso of user 932. Strap 940 is fixedly attached to sensor assembly 936 at attachment mechanism 954. Strap 938 is fixedly attached on one end to arm sling 930 at wrist area 948, e.g., by way of a rivet, Chicago binding post or the like. Sensing assembly 936 includes a mounting passage 942 that accommodates passage of the free end of strap 938 in a “looping” fashion relative to sensing assembly 936, thereby allowing the user 932 to pull on the free end of strap 938 to cinch the arm sling to shoulder strap 940. The cinching action creates a tension in the strap that facilitates support for immobilization of the arm. The sensing assembly 936 provides advantageous monitoring and feedback functionality according to the present disclosure. The arm sling 930 can also include a first strap 950 to secure the sling to the user's 932 upper arm and a second strap 944 to secure the arm sling 930 to the user's 932 forearm.

In another embodiments, the sensing assembly can be disposed in the elbow area 946 of the elbow brace 934. The sensing assembly can be configured to measure range of motion of the elbow area 946.

With reference to FIG. 9F, a front portion of an exemplary orthotic foot brace 956 is shown secured to a user 986. Foot brace 956 includes an outer hard shell 962 and an inner soft-material lining 960 with first and second portions 988, 990 that overlap in an interface region 974. Of note, although the exemplary embodiment of FIG. 9F depicts an overlap of first and second portions 988, 990, alternative foot brace implementations may instead define a “gap” between first and second portions 988, 990. Thus, the overlap region 974 may take the form of a “gap” between cooperative portions of the disclosed foot brace, and references to “overlap regions” and “gaps” should be understood to embrace the relative positioning of the first and second portions, whether such relative positioning defines spacing, overlap or even side-by-side juxtaposition.

A plurality of straps are mounted with respect to foot brace 956 to facilitate securement thereof with respect to the user's lower leg, ankle and foot within the soft-material lining 960. In particular, exemplary foot brace 956 includes first strap 958, second strap 964, third strap 968, a forth strap 970 and a fifth strap 972 attached to hard shell 962. As will be readily apparent to persons skilled in the art, the present disclosure is not limited to brace implementations that include four straps, or to brace implementations wherein the straps are located on the front face of the brace. Rather, the present disclosure may be implemented with fewer or greater numbers of straps without departing from the spirit or scope of the present disclosure, or to rear and/or side positioning of straps. Positioning of the straps on the front face of the foot brace may be preferable in specific usage environments, e.g., for user to easily access the strap adjustments. With further reference to FIG. 9F, each of the straps 958, 964, 968, 970 and 972 is fixedly mounted to one side or the other of the hard shell 960. More particularly, first strap 958 is fixedly mounted with respect to hard shell 960 by attachment mechanism 976, second strap 964 is fixedly mounted with respect to hard shell 960 by attachment mechanism 978, third strap 968 is fixedly mounted with respect to hard shell 962 by attachment mechanism 980, the fourth strap 970 is fixedly mounted with respect to hard shell 962 by attachment mechanism 984 and fifth strap 972 is fixedly mounted with respect to hard shell 962 by attachment mechanism 982. In the disclosed embodiment, second strap 964 releasably cooperates with sensing assembly 966 that is mounted with respect to hard shell 962 at attachment mechanism 978. Sensing assembly 966 provides advantageous monitoring and feedback functionality according to the present disclosure, as described in greater detail below. Similarly, second strap 964 releasably cooperates with sensing assembly 966 that is mounted with respect to first portion 988 of brace 956 by mounting strap 992. Sensing assembly 966 also provides advantageous monitoring and feedback functionality according to the present disclosure, as described in greater detail below.

In the exemplary embodiment of FIG. 9F, each of the mounting straps 958, 964, 968, 970 and 972 is fixedly mounted to the left side of hard shell 962. However, the present disclosure in not limited to the “same side fixation” arrangement depicted in FIG. 9F, and brace-based systems may be implemented according to the present disclosure wherein the straps are fixedly mounted on alternating sides of the hard shell 962 in an “opposed fixation” arrangement without departing from the spirit or scope hereof. In fact, the opposed fixation arrangement of the straps relative to the hard shell 962 of foot brace 956 may improve the stability and/or ease with which the foot brace may be brought into a desired orientation by the user.

With further reference to FIG. 9F, sensing assembly 966 includes a mounting passage that accommodates passage of second strap 964 in a “looping” fashion relative to sensing assembly 966, thereby allowing the user 986 to pull on the free end of strap 964 to cinch the left side relative to right side of hard shell 962, thereby decreasing the gap between left side relative to right side of hard shell 962. In implementations wherein a gap is defined between the first and second portions of a brace, the cinching operation will serve to reduce the gap and/or bring the two portions into a juxtaposed or overlapping orientation. Once cinched to a desired degree, second strap 964 is generally adapted to be detachably fixed in the desired position, e.g., by way of cooperative Velcro™ interaction in the overlapping region of strap 946. Alternative fixation mechanisms may be employed to secure strap 964 in its cinched orientation, as will be readily apparent to persons skilled in the art. Looping, cinching and fixation mechanisms are generally provided with respect to first strap 958, third strap 964, forth strap 970 and fifth strap 972, thereby permitting the user to bring the left side and the right side of hard shell 962 into a desired approximation.

In conventional foot brace systems, the desired cinched relationship between the left side and the right side of hard shell 962 is inexactly established. For example, a physician or other health care provider may apply a mark, e.g., a line, on some aspect of the foot brace system to designate the desired spatial relationship of the left and right sides of hard shell 962, when in use. The user 956 then strives to bring the hard shell sides into alignment with the designated marking, subject to visibility limitations, parallax issues and difficulties in applying the requisite force to achieve the desired brace orientation. Moreover, conventional foot brace systems provide no ability to monitor the brace orientation over a period of use and/or identify changes to applicable parameters, e.g., the user's anatomy, that may impact on the accuracy of the initial “marking” provided by the physician or other health care provider. The disclosed systems and methods overcome the noted limitations and shortcomings of existing foot brace systems.

With reference to FIG. 9G, a prosthetic leg 994 that includes an upper leg portion 1006, a lower leg portion 1008 and a foot portion 1010 is shown with a cone-shaped cavity 1002 attached at the top 1004 of the upper leg portion. The cone-shaped cavity 1002 is made of a pliable material that can receive and secure the stump portion of a leg that has been amputated, thus providing the user with an artificial leg. The pliable cone-shaped cavity 1002 is tightened around the stump with a conventional “loop strap” system similar to that described in FIG. 9A.

Sensor assembly 996 is attached to an end 1000 of strap 998. The end of sensor assembly 996 is attached to cone-shaped cavity 1002. Again, the sensor assembly 996, in-line with strap 998, permits a continuous measure of parameters such as, but not limited to, tension, position, pressure and temperature with advantageous monitoring and feedback functionality according to the present disclosure.

With reference to FIG. 9H, a front view of an alternative embodiment of the brace sensor mechanism is shown with a prosthetic arm 1014 secured to the torso of a user 1012. Prosthetic arm 1014 includes a conventional circular strap 1030 that is fixedly attached to the shoulder area 1032 of the prosthetic arm 1014. A second conventional strap 1020 is fixedly attached to the circular strap 1030 on the backside (not shown) and wraps around the back and under the user's natural arm 1022, and then extends across the user's front chest 1024. The front end 1026 of strap 1020 is attached to sensor assembly 1018. One end 1028 of sensor assembly 1018 is releasably attached to a connector strap 1016, which is fixedly attached to circular strap 1030. The tension in the straps 1030, 1020 and 1016 is adjusted to achieve a fitting of the prosthetic arm 1034 that is physically safe, secure and comfortable to the user. The sensor assembly 1018, positioned in-line with the conventional straps 1020 and 1030 permits a measure of the tension in the straps. The sensor assembly 1018 in FIG. 9H provides advantageous monitoring and feedback functionality according to the present disclosure, as described in greater detail below.

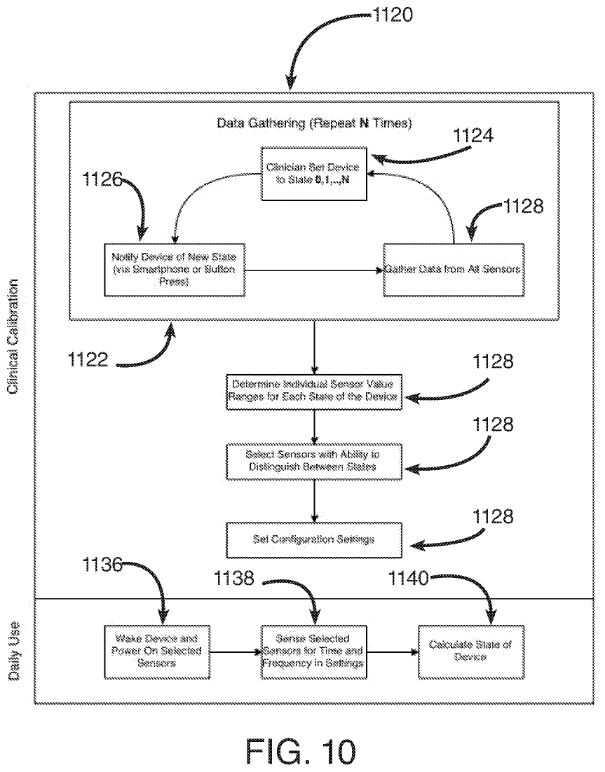

FIG. 10 provides a schematic flowchart 1120 of exemplary data flow within the orthotic or prosthetic device in clinical calibration of the device and daily use of the device according to implementations of the present disclosure. While in clinical calibration the device may gather data in a loop 1122. While in the loop the device can be set to gather data a set predetermined amount of iterations (step 1124). In step 1126, the device can be notified of a new state, via smartphone or other interaction with the device. In step 1128 the device can gather data from all the sensors and the device can repeat the loop until the device reaches the set predetermined iterations.

In step 1130, the device can determine the individual sensor value ranges for each state of the device. For example, in order to determine the compliance and quality of orthotic and prosthetic device, the orthotic or prosthetic device can collect data associated with the measured metric/parameter while a user is at a clinic and compare the data associated with the measured metric/parameter collected at the clinic with data associated with the measured metric/parameter collected at a different predetermined time period in the past. The comparison can show degradation of the orthotic and prosthetic device or non-compliance by the user at a previous predetermined time period. The orthotic or prosthetic device can use the collected data to determine different states of the device. The different states can be but are not limited to: the device turned off, device is worn correctly, and the device is loose. A personalized comparative value can be established for each patient and each sensed metric/parameter. This information can also be used to optimize the sensing characteristics of the individual sensors by determining which of the sensed values changes between states and the frequency at which the sensor data must be recorded to capture the change between states. In step 1132 the sensors can be selected with the ability to distinguish between the states. In step 1134, the configuration settings of the device can be set. While in daily use, in step 1136 the device can “wake” and power on selected sensors. In step 1138, the sensors can sense the time and frequency in the settings. In step 1140, the sensors can calculate the state of the device.

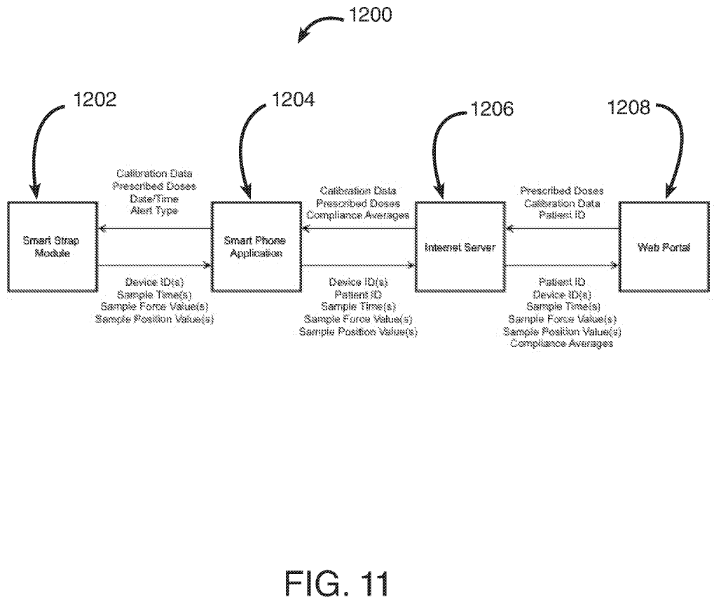

FIG. 11 provides a schematic flowchart 1200 of exemplary data flow according to implementations of the present disclosure. The flow of data according to the present disclosure generally begins in the device/system (“Smart Strap Module 1202”), where the force and/or position sensors are located. The device generally measures and/or captures sample time(s), sample force value(s) and sample position value(s). Data may be shuttled to a Bluetooth module for transmission to external devices, e.g., a computer or smartphone interface (“Smart Phone Application 1204”). The Smart Phone Application 1204 can in turn communicate with an Internet Server 1206 that then communicates with a Web Portal 1208. Communications may proceed in the opposite direction, i.e., originating from the Web Portal 1208 and ultimately reaching the Smart Strap Module 1202, e.g., prescribed does, calibration data, and patient ID. Thus, the Web Portal may be associated with an external device to facilitate transfer of the data to a web-based database and associated processing capabilities. In addition, the Web Portal 1208 may support access and use of the data by interested parties, e.g., physicians, patients, parents and operational centers.

Thus, the disclosed device components may include sensors that are adapted to monitor and/or measure position (e.g., the resistance and magnetic systems described above) and/or tension/force (e.g., the strain gauge systems described above). The parameters measured by the disclosed sensors may be processed by a microcontroller associated with a circuit board that generally includes programming to drive the features and functions described herein. The device components also generally include appropriate data storage, e.g., a memory card such as a Micro-SD (secure digital) non-volatile memory card.

Once the microcomputer receives information from the sensor(s), the microcomputer may be programmed to actuate a variety of immediate feedback mechanisms, e.g., to notify the patient/user when certain conditions are met. Feedback mechanisms may be selected by the patient/user and customized depending on applicable variables, e.g., the type of device (e.g., prosthetic or orthotic device), the needs of the patient/user, the age/maturity of the patient/user and the like.

The device components also generally include one or more features/functions that are adapted to provide immediate feedback to users/caregivers with respect to brace use and performance. Thus, as described above, the disclosed system may include device components that are adapted to generate and deliver light signals, haptic/vibratory signals and/or sound-based signals. For example, RGB LED lights may be adapted to deliver feedback to the patient/user by changing color, intensity and/or the number of lights that are illuminated. In exemplary embodiments, the color of illumination light and/or aspects of the illumination (e.g., blinking rate) may be used to communicate information concerning the quality of device usage, as described with reference to previous embodiments. For example, a green LED may be illuminated if the quality of usage is good, a red LED may be illuminated if the quality of use is poor, and a yellow LED may be illuminated if the quality is of intermediate quality. Similarly, rapidity at which the LED is blinked may be used to signal proximity to a desired (or undesired) position of the brace. Auditory feedback may be delivered in various ways, e.g., a piezoelectric buzzer may be used to alert a patient/user of a sensed condition even if the patient/user is not looking at the device. Haptic/vibratory feedback may be particularly valuable to patients/users, e.g., when the device is located so as to be out of the user's line of sight (e.g., adjacent a patient's back), which means that the patient will not be able to see visual feedback associated with the device. Haptic/vibratory feedback may also be generated and delivered in a manner that is not apparent to others in the vicinity, thereby preserving the privacy of the patient/user.

Still further, device components associated with the present disclosure generally include elements that are adapted to support data transmission, e.g., a Bluetooth module. For example, the microcontroller of the disclosed system may be adapted to relay stored data to the Bluetooth module for output in a serial stream that can be received and read by smartphones, computers and other Bluetooth-enabled electronic devices/systems. Power is generally delivered to the disclosed device components by appropriate battery technology, e.g., rechargeable lithium polymer battery. Charging of the disclosed battery may be accomplished by way of a micro-USB connection and/or internal charging circuitry associated with the disclosed system. Information generated by the disclosed device components are advantageously transmitted, e.g., by way of a Bluetooth communications, to external processing and/or data storage units.

Bluetooth transmissions may be employed to transmit information that is sensed and processed by the device components to external systems, such as an external computer and/or smartphone.

In addition, the information that is transmitted from the disclosed device components may be routed to a network-based system, such as an online database and associated processing functionality. In exemplary implementations, the information that is collected by the device components associated with a device may be routed to an application that permits access by a physician and/or other health care provider, thereby permitting condition-related assessments and adjustments to be undertaken in a timely and effective manner without the need for frequent office visits by the patient. Interaction with and analysis of the data generated by the disclosed systems may be facilitated by appropriate user interfaces that are programmed to deliver user-friendly information display and associated processing tools. Different user interfaces may be provided for different user groups, e.g., patients and physicians/health care providers.

The information that is transmitted to external systems and the immediate feedback generated by the device components, e.g., visual, haptic and/or sound communications, may benefit the patients, their parents (and other caregivers) and doctors (and other health care providers). Still further, research organizations and/or central monitoring organizations may have access to or otherwise receive information that is generated according to the present system.

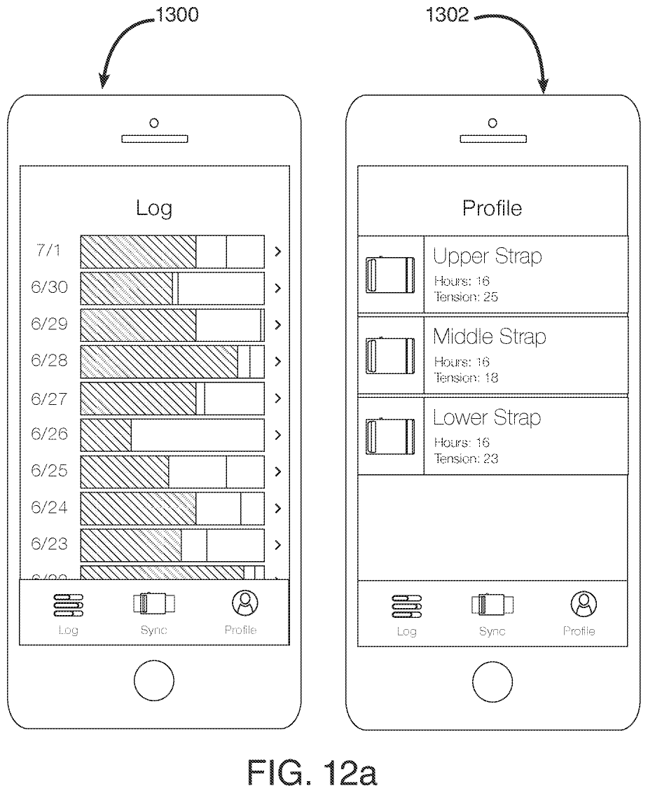

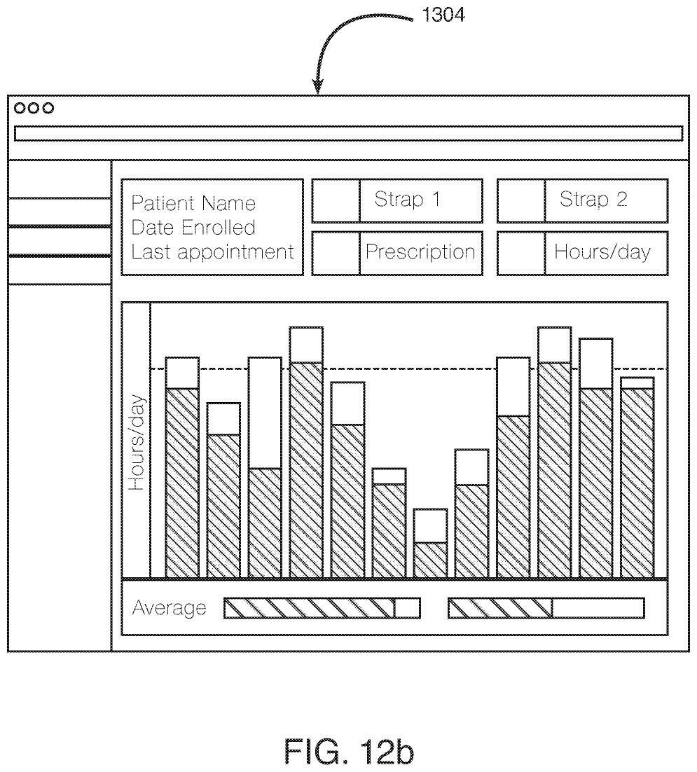

With reference to FIGS. 12A-12B, exemplary data displays that are supported by the monitoring/measuring and feedback systems of the present disclosure are illustrated, as follows:

FIG. 12A depicts an exemplary screenshots 1300 and 1302 of a mobile application showing long-term feedback via bar graphs and a prescription display according to the present disclosure. As shown in screenshot 1300, along at top of the screenshot, a “log” link is provided that allows review of the user's usage log. Below the “log” link, the display shows exemplary device usage for a series of days (7/1-6/23), including specifically the number of hours of device usage and the tightness relative to prescribed level (as a percentage). As shown in screen shot 1302, along the top “profile” link is provided that allows the review of the user's usage of each strap. For example, the user can see the number of hours each strap was strapped on and the amount of tension provided to each strap.

FIG. 12B depicts a further exemplary screenshot 1304 that shows a web application for clinicians showing long-term data display and summaries, as well as prescription. The screenshot 1304 depicts a patient dashboard. The patient dashboard includes patient bio, data associated with various braces, prescription, feedback information and a bar graph of the usage data with the usage hours along the y-axis and the days along the x-axis. The patient bio can include date of birth, type of brace, and patient number. The data associated with the braces can include the data collected for different straps, i.e. lower and upper including position and tension information. The bar graph will indicate the total hours the device was worn along with the total hours the device was correctly worn for each day as with reference to the prescribed hours. As noted above, the modular units of the present disclosure may be used in conjunction with various prosthetic/orthotic devices and may be used to measure/sense various metrics/parameters. The measured/sensed data may be used to calculate various performance- and/or usage-related values, e.g., step count, activity, range of motion, orientation, and other measurements. The modular units may be associated with strap(s), belt(s), webbing, ratchet(s) and other tensioning devices/systems.

In some embodiments, the orthotic or prosthetic device can have a learning mode configured to compare data associated with the measured metrics/parameters collected at a particular predetermined time period with data associated with the measured metrics/parameters at another predetermined time periods in the past.

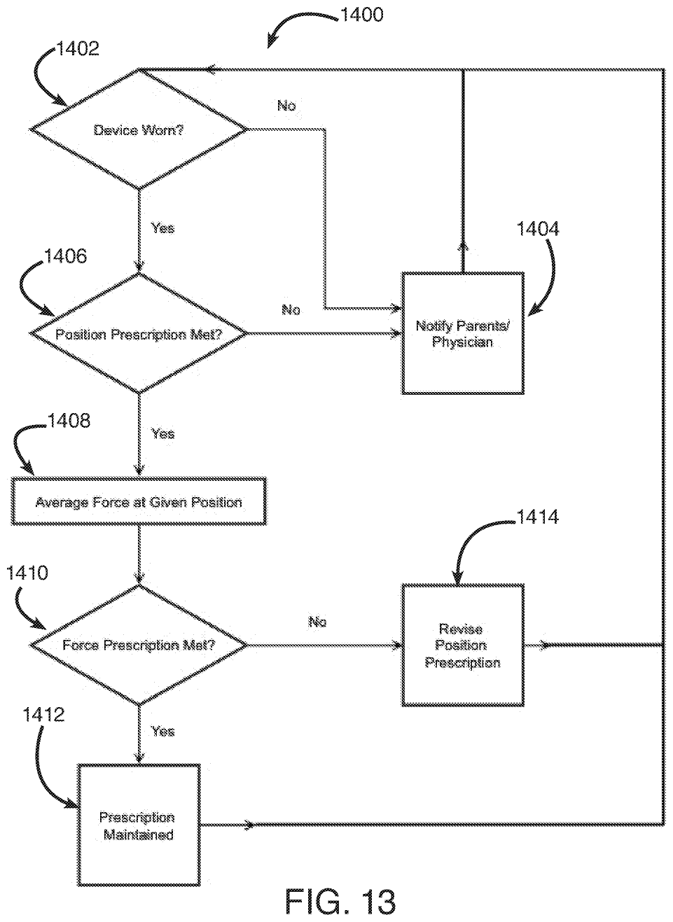

FIG. 13 provides an exemplary flowchart 1400 that illustrates a sequence of steps by which the disclosed system/method may be determine quality and compliance of the orthotic or prosthetic device use. Alternative and/or additional metrics may be measured/calculated with respect to device use utilizing the measured data/forces. Thus, the system/method may determine whether the device is being worn (Step 1402). If not, the parents and/or physician may be notified (Step 1404). Conversely, if the device is being worn, the system/method determines whether the prescription as to device positioning is being satisfied (Step 1406). If not, the parents and/or physician may be notified (Step 1404). If so, the average force at the applicable device position is determined (Step 1408). Based on the average force determination, the system/method determines if the prescription as to force is being met (Step 1410). If not, the position prescription is revised to deliver the desired force level (Step 1414). Conversely, if the force prescription is being met, then the prescription level is maintained (Step 1412) and the system/method rechecks quality/compliance, as and when prompted, e.g., based on a preset frequency schedule. As noted above, additional/alternative metrics may be measured, calculated and reported according to the present disclosure.

The present disclosure has been described with reference to various exemplary implementations and embodiments of the advantageous systems and methods for monitoring and/or measuring parameters related to the use of devices, e.g., compliance and quality of orthotic or prosthetic device usage, step count, activity, range of motion, orientation, or other measurements. However, the present disclosure is not limited by or to the exemplary implementations and embodiments described herein. Rather, the systems and methods of the present disclosure are susceptible to many alternative implementations and embodiments without departing from the spirit or scope provided herein, as will be readily apparent to persons skilled in the art. Accordingly, the present disclosure expressly encompasses and embraces such alternative implementations and embodiments within its scope.

1. A system for measuring one or more parameters, comprising:
a. an adjustable implantable device configured and dimensioned to be mounted with respect to an anatomical location, said adjustable implantable device including a tensioning mechanism that includes (i) at least one of a strap, belt, webbing or ratchet mechanism, and (ii) a cooperative structure that is configured and dimensioned to cooperate with the at least one of a strap, belt, webbing or ratchet mechanism to secure the adjustable implantable device relative to the anatomical location; and
b. at least one sensor system mounted directly to the tensioning mechanism, said at least one sensor system including an inductive sensor and a conductive material spaced from said inductive sensor;
wherein the at least one sensor system measures the position of the adjustable implantable device relative to the anatomical location.

2. The system according to claim 1, wherein the tensioning mechanism includes at least one strap, and wherein the cooperative structure includes a device loop that is sized to receive a free end of the at least one strap there through.

3. (canceled)

4. (canceled)

5. The system according to claim 1, wherein the conductive material and the inductive sensor of the at least one sensor system interact to generate one or more signals that may be used to determine a distance between two points on the adjustable implantable device.

6. The system according to claim 1, further comprising electronic elements including a power source and a processing element associated with and in communication with the at least one sensor system for processing information generated by the inductive sensor.

7. The system according to claim 6, wherein the processing element is configured to determine at least one of compliance of the adjustable implantable device use, quality of adjustable implantable device use, step count, activity, range of motion, orientation, a distance between two points on the adjustable implantable device, position of the adjustable implantable device, tightness of the implantable device or other user-related measurement based on information generated by the at least one sensor system.

8. The system according to claim 6, further comprising a transmitter for communicating the information generated by the at least one sensor system to an external device.

9. The system according to claim 4, further comprising one or more signaling elements that are adapted to provide at least one of real-time aural, visual and haptic feedback as to use of the adjustable implantable device.

10. A method for measuring one or more parameters associated with use of an adjustable implantable device, comprising:
a. providing at least one sensor system mounted with respect to the adjustable implantable device, said at least one sensor system including an inductive sensor and a conductive material spaced from said inductive sensor;
b. providing at least one tensioning mechanism configured and dimensioned to interact with the at least one sensor system,
c. determining at least one parameter associated with adjustable implantable device usage based on measurement of the position of the adjustable implantable device,
wherein the adjustable implantable device is configured and dimensioned to be mounted with respect to an anatomical location, said tensioning mechanism includes (i) at least one of a strap, belt, webbing or ratchet mechanism, and (ii) a cooperative structure that is configured and dimensioned to cooperate with the at least one of a strap, belt, webbing or ratchet mechanism to secure the adjustable implantable device relative to the anatomical location; and
wherein the at least one sensor system is mounted directly to the tensioning mechanism.

11. The method according to claim 10, further comprising, processing the at least one parameter using electronic elements including a power source and a processing element associated with and in communication with the adjustable implantable device.

12. The method according to claim 11, wherein the processing element is configured to determine at least one of compliance of the adjustable implantable device use, quality of adjustable implantable device use, step count, activity, range of motion, orientation, a distance between two points on the adjustable implantable device, position of the adjustable implantable device, tightness of the adjustable implantable device or other user-related measurement based on information generated by the at least one sensor system.

13. (canceled)

14. The method according to claim 10, wherein determining the at least one parameter further comprises determining a distance between two points on the adjustable implantable device knee brace based on signals produced by the inductive sensor mounted on the adjustable implantable device.
